(19) United States
(12) Patent Application Publication
(10) Pub. No.: US 2020/0178580 A1
(43) Pub. Date: Jun. 11, 2020

(54) MALT SPROUTS EXTRACTS AND THEIR USES

(71) Applicant: MALTEUROP GROUPE, REIMS (FR)

(72) Inventors: Marie-Jane FALLOURD, BEZANNES (FR); Sihame CHAFIL, FESTIEUX (FR); Jean-Luc BARET, LEOGNAN (FR)

(21) Appl. No.: 16/470,069

(22) PCT Filed: Dec. 18, 2017
(86) PCT No.: PCT/EP2017/083403

§ 371 Date: Jun. 14, 2019

(30) Foreign Application Priority Data
Dec. 16, 2016 (EP) 16306713.5
Dec. 22, 2016 (EP) 16206452.1

(51) Int. Cl. A23L 7/25; C12P 21/06; C12N 1/16; C12N 1/20; A23L 33/105; A23L 33/13; A23L 33/175; A23L 33/18; A23L 27/00; A23K 20/142; A23K 20/153; A23K 10/12

(52) U.S. Cl. CPC A23L 7/25; C12P 21/06; C12N 1/16; C12N 1/20; A23L 33/105; A23L 33/13; A23V 2002/00; A23L 33/18; A23L 27/88; A23K 20/142; A23K 20/153; A23K 10/12; A23L 33/175

(57) ABSTRACT

Disclosed are compositions containing malt sprouts fraction extracts, their process of preparation and their uses.

The present invention relates to malt sprouts extracts and their uses.

### BACKGROUND OF THE INVENTION

It is well known that nucleotides have a fundamental role in all living cells, as being involved in genetic information, protein biosynthesis and cell metabolism. Nucleotides include nitrogenous bases, i.e. purines (adenine, guanine) and pyrimidines (cytosine, thymine), which are constituents of DNA. Uracil replaces thymine in RNA.

The purine nucleotides are formed through a complex biosynthetic pathway involving some amino acids as precursors of the purine nucleus. Pyrimidine nucleotides synthesis involves also an amino acid and ribose phosphate.

The complexity of the purine nucleotides synthesis pathway, leads the cell to recover purines from purine nucleotides degradation. As a consequence, purines are largely recycled to form new nucleotides. This recycling process requires much less energy than the purine nucleotides synthesis, as processing within one reaction, and therefore provides an advantage to the cell. Although the pyrimidine nucleotides biosynthesis pathway is simpler than the purine nucleotides one, the availability of pyrimidines also provides an advantage to the cell.

Furthermore, cell cultures generally require suitable media consisting of different carbon and nitrogen sources, mineral salts, nutrients and growth factors. Various components or ingredients provide simple or complex sources for the formulation of the media. Among them, protein hydrolysates have been used for many years. Yeast extracts, plants or animal peptones are good sources of total nitrogen (Nt) and free amino nitrogen FAN, they also contain residual carbohydrates, minerals, and different anions and cations (BD BioNutrients™ Manual, Becton Dickinson 4^{TH} edition). These ingredients are generally presented in the form of powders or liquid preparations, fully soluble in water, developed for some industrial uses, and generally ready for use for various laboratory applications (e.g. media for microbiological controls). However, they are relatively expensive and contribute significantly to the costs of the formulation of media compositions.

Malt sprouts are considered as sources of many different products for various purposes Similarities exist in methods or processes which are divulgated in the prior art, because the unit operations mainly consist in putting in contact a liquid phase (often an aqueous solution) and a solid phase (malt sprouts) followed by the processing of the resulting mixture to separate the liquid phase from residual solids, then optionally followed by down streams treatments.

The main differences between those processes and methods can be found in the operating conditions required to obtain the targeted products. Those differences in operating conditions lead to the characteristics of the products described in the prior art. However, the presence of nucleic acids and Nucleic acid derivatives (DAN) in prior art compounds was so far unknown.

Nucleic acids are complex macromolecules with high molecular weight, which can be chemically or enzymatically hydrolyzed in a multitude of products including oligonucleotides, nucleotides, nucleosides and elementary constitutive molecules. A total hydrolysis leads to nucleic bases, phosphate and pentoses either deoxyribose (from DNA) or ribose (from RNA).

The techniques of the prior art do not solve the problem. Nucleic acid hydrolysis is only partial and does not allow to obtain high concentration of free nucleic bases.

The detailed compositions of main protein hydrolysates such as animal-free peptones, yeast extracts and meat peptones can be found in the technical literature for instance in BD Bionutrients TM Technical manual. However, to the best of our knowledge, it can be noted that no indication on the characterization and contents of nucleic acid derivatives (DAN) is available for those products. There is a lack of crucial information on DAN which limits the possibilities to improve the technical performances of the culture media and to master the costs of formulations. It can also be noted that the analytical determination of DAN requires specialized analytical methods and tools, which are rather techniques for research purpose than routine ones. The inventors carried out the analytical determination of DAN and more particularly the nucleic bases purines and pyrimidines (PUPY) in some available protein hydrolysates whose results are reported in the following table:

| | | | | | | Ratio |
|---|---|---|---|---|---|---|
| | | Total | Total | Total | Total | PUPY/ |
| | Supplier/ | nitrogen | FAN | DAN | PUPY | DAN |
| | product ref | g/kg dm | g/kg dm | g/kg dm | g/kg dm | % |
| Soy | Solabia/A1603 | 98.9 | 24.7 | 1.6 | 0.4 | 22.3 |
| peptone | | | | | | |
| Meat | ABS laboratoire/ | 137.3 | 19.9 | 2.4 | 0.9 | 37.3 |
| peptone | AEB171206 | | | | | |
| Poly- | BD/211910 | 126.1 | 12.2 | 4.6 | 2.0 | 44.4 |
| peptone | | | | | | |
| Yeat | ABS laboratoire/ | 114.6 | 36.1 | 7.9 | 1.6 | 20.2 |
| extract | AEB171106 | | | | | |
| Yeast | Bio Springer/ | 109.3 | 18.4 | 8.9 | 1.4 | 15.7 |
| extract | 0251-PWL | | | | | |

Although the manufacture of protein hydrolysates has greatly improved, resulting available products still have limitations to cover the different nitrogenous matter needs of cells in various applications. There is thus a need to develop new types of nitrogen sources for culture media for applications such as cell culture and fermentation process, not only to reduce costs associated with their use but also to introduce highly functional ingredients with improved properties in cell cultures.

Moreover, it is well known that nucleotide-rich protein hydrolysates (e.g. nucleotide-rich yeast extracts) have interesting organoleptic properties, notably the ability to enhance flavor in food products.

So far, co-products of the malting industry are mostly valued in Feed industry. Indeed, the study “Enquête sur les gisements et la valorisation des co-produits issus de l'Agro-Industrie”‘, Ademe-Reseda, 2008, states that from 1,656 MT of barley transformed in France in 2007, the malting process produced 1,325 MT of malt and generated 77 000 T of co-products among which 56 000 T of malt sprouts and 21,000 T of orgettes.

### DETAILED DESCRIPTION OF THE INVENTION

One of the aims of the invention is to provide new types of DAN-enriched extracts with high proportion of PUPY, as nitrogen sources, starting from malt sprouts fractions.

One of the aims of the invention is to provide a process enabling to hydrolyze nucleic acids contained in malt sprouts fractions to obtain high concentrations of nucleic bases in malt sprouts fraction extracts and to obtain concomitantly quantitative yields of soluble matter in those extracts.

One of the aims of the invention is to provide a process enabling to prepare protein mixture containing malt sprouts fraction extract and additional nitrogen compounds, starting from malt sprout fractions and primary protein sources both engaged in this same process.

One of the aims of the invention is to provide a new use of DAN-enriched malt sprouts fraction extracts with high proportion of PUPY, as a nitrogen source and/or sugar source and/or protein source and/or in a culture or fermentation process, or in the culture of cells, or as a flavor enhancer.

One of the aims of the invention is to provide a new use of co-products of the malting industry, with a better valorization as high value-added products.

The first object of the invention is a composition containing a malt sprouts fraction extract, wherein the composition comprises at least 0.5%, and less than 98% of dry matter by total weight of the composition, and characterized at least by:
Total Nitrogen content from 40 to 140 g/kg of dry matter
Free Amino Nitrogen content from 6 to 60 g/kg of dry matter, and
Nucleic Acid Derivatives such as purines and pyrimidines bases in an amount from 3 to 25 g/kg of dry matter.
PUPY content from 1 to 20 g/kg of dry matter
Ratio PUPY/Nucleic Acid Derivatives higher than 20%.

The above-mentioned composition according to the invention is characterized by a ratio PUPY/Nucleic Acid Derivatives from 20 to 100%, in particular from 20 to 30%, from 20 to 40%, from 20 to 50%, from 20 to 60%, or from 20 to 70%.

Starting from malt sprouts, it was not known whether the concentration of nucleic bases potentially extracted from said material would be high enough to produce the desired effect in cell cultures. It was also not known how to proceed to extract said hypothetical appropriate concentration of nucleic bases from malt sprouts.

The above-mentioned composition containing a malt sprouts fraction extract can be:
a composition consisting exclusively a malt sprout fraction extracts
a composition comprising at least malt sprouts fraction extracts and at least one additional ingredient selected from protein hydrolysates
a composition such as obtained by the process of preparation of a protein mixture containing malt sprouts fraction extracts and additional nitrogen compounds,

In the sense of the invention, the term “malt sprouts” includes the rootlets, the acrospires, the husks, the dust and mixtures thereof and corresponds to the co-products generated during the malting process. Malt sprouts are represented in FIG. 1 showing the rootlets, the acrospire and the husk.

In the sense of the invention, the term “malt sprouts fraction” refers to a specific and selected fraction of malt sprouts obtained by sieving (from 0.5 to 2.4 mm of width, preferably from 0.5 to 2 mm) or by air blowing or combination of both. These fractions, used as a starting material, offer a better performance in the processes and have a higher protein content as well as a better processing ability than malt sprouts.

In the sense of the present invention, the terms “malt sprouts fraction”, “isolated malt sprouts fraction” and “malt fraction” are equivalent and can be used interchangeably.

The term “malt sprouts fraction extracts” according to the invention encompasses the diluted malt sprouts fraction extracts as juice, also named “malt sprouts juice” or “juice of malt sprouts”, the concentrated malt sprouts fraction extracts as juice (liquid or slurry form) and the powder malt sprouts fraction extracts (solid form) as further defined.

Malt sprouts fraction extracts encompasses the products, containing water soluble components from malt sprouts or malt sprouts fractions, such as nitrogen, free amino nitrogen (FAN) and nucleic acid derivatives (DAN), these components being initially present in these matters and obtained by diffusion, or solubilized by hydrolysis of some initially insoluble components of these matters, as hereafter detailed. Hydrolysis results from the action of endogenous or exogenous enzymes.

Malt sprouts fraction extracts exist under different forms during processing and according to final product formulations.

A primary malt sprouts fraction extract is the diluted aqueous phase (0.5 to 10% dm) obtained in the front part of the processes (before concentration), it contains the dissolved components and potentially some residual suspended solids.

A secondary malt sprouts fraction extract is a concentrated extract (10 to 98% dm) either in liquid form as a syrup or dough, or solid form as a dry powder.

In the sense of the present invention, the terms “malt sprouts fraction extracts”, “malt fraction extracts” and “malt extract fraction” are equivalent and can be used interchangeably.

As an indicative composition of malt sprouts fractions, in particular barley malt sprouts fractions, used as initial raw material for the extraction process according to the invention, it may advantageously contain:
92 a 98% of Dry matter (d.m)
22.5-37% d.m. of Crude protein
43-52% d.m. total fibers from which 40-50% insoluble fibers (cellulose, hemicellulose, lignin) and 2-5% of soluble fibers;
7-12% d.m. total carbohydrates from which 4-7% starch and 4-6% sugars
<1% d.m fats
5-7% d.m ash.

According to a particular embodiment of the invention, the malt sprouts fractions contain 23, 25, 27, 29, 31, 33, 35 or 37% d.m. of Crude protein.

The term “Total Nitrogen content” is the sum of nitrogen bound in organic substances, nitrogen in the form of ammonia (NH3-N) and ammonium (NH4+−N). The total nitrogen content according to the invention is determined by a quantitative method, the Kjeldahl method (Zeitschrift fur Analytische Chemie, Herausgeber Dr. C. Remigius Fresenius. 22. armée, éditions C. W. Kreidels Verlag 1883. p. 366-382, J. Kjeldahl).

The term “Free Amino Nitrogen (FAN)” content is defined as the sum of the individual amino acids, ammonium ions, and small peptides (di- and tripeptides) in wort or liquid phase. FAN is determined by the EBC (European Brewery Convention) method 4.10.

The term “Nucleic acid derivatives (DAN)” refers to intermediate and final products coming from the hydrolysis process of nucleic acids (DNA: DeoxyriboNucleic Acid, and RNA=RiboNucleic Acid). DAN is measured by the method described in the following article: An Ion-Pairing High-Performance Liquid Chromatographic Method for the Direct Simultaneous Determination of Nucleotides, Deoxynucleotides, Nicotinic Coenzymes, Oxypurines, Nucleosides, and Bases in Perchloric Acid Cell Extracts (Donato Di Pierro2, Barbara Tavazzi, Carlo Federico Perno, Marco Bartolini, Emanuela Balestra, Raffaele Calio', Bruno Giardina,* and Giuseppe Lazzarino)—Department of Experimental Medicine and Biochemical Sciences, University of Rome “Tor Vergata,” Via Tor Vergata 135,00133 Rome, Italy; *Institute of Chemistry and Clinical Chemistry and “Centro CNR per la Chimica dei Recettori,” Catholic University of Rome “Sacro Cuore,” Rome, Italy; and †Institute of Biochemical and Pharmacological Sciences, University of Catania, Catania, Italy—1995).

DAN are constituted of a complex mixture of:
Nucleotides, which form the basic structural unit of nucleic acids, composed of:
   a phosphate group,
   a purine nitrogenous base (adenine, guanine) or a pyrimidine nitrogenous base and cytosine, thymine, uracil),
   and a pentose sugar (a ribose for RNA and a deoxyribose for DNA).

| Base | Ribonucleoside-5′-monophosphate | Deoxyribonucleoside-5′-monophosphate |
|---|---|---|
| Adenine | Adenosine-5′-monophosphate = AMP | Deoxyadenosine-5′-monophosphate = dAMP |
| Guanine | Guanosine-5′-monophosphate = GMP | Deoxyguanosine-5′-monophosphate = dGMP |
| Uracil | Uridine-5′-monophosphate = UMP | Deoxyuridine-5′-monophosphate = dUMP |
| Cytosine | Cytidine-5′-monophosphate = CMP | Deoxycytidine-5′-monophosphate = dCMP |
| Thymine | Thymidine-5′-monophosphate (rare) | Deoxythymidine-5′-monophosphate = dTMP |

Nucleosides, which consist simply of a nitrogenous base linked to a pentose sugar (either ribose or deoxyribose) by a glycosidic bond. According to the invention nucleosides include cytidine, uridine, adenosine, guanosine, thymidine and inosine.

| Base | Ribonucleoside | Deoxyribonucleoside |
|---|---|---|
| Adenine | Adenosine | Deoxyadenosine |
| Guanine | Guanosine | Deoxyguanosine |
| Uracil | Uridine | Deoxyuridine |
| Cytosine | Cytidine | Deoxycytidine |
| Thymine | 5-methyluridine | Thymidine |

Nitrogenous bases, named PUPY (for PUrines and PYrimidines) according to the invention, are organic molecules with a nitrogen atom that has the chemical properties of a base. The main biological function of a nitrogenous base is to bond nucleic acids together. According to the invention nitrogenous bases include adenine, guanine, cytosine, thymine, uracil and hypoxanthine. Hypoxanthine is a naturally occurring purine derivative. It is occasionally found as a constituent of nucleic acids.

Nitrogenous bases are typically classified as the derivatives of two parent compounds, pyrimidine and purine. Purine nitrogenous bases are adenine and guanine and pyrimidine nitrogenous bases are cytosine, thymine, and uracil.

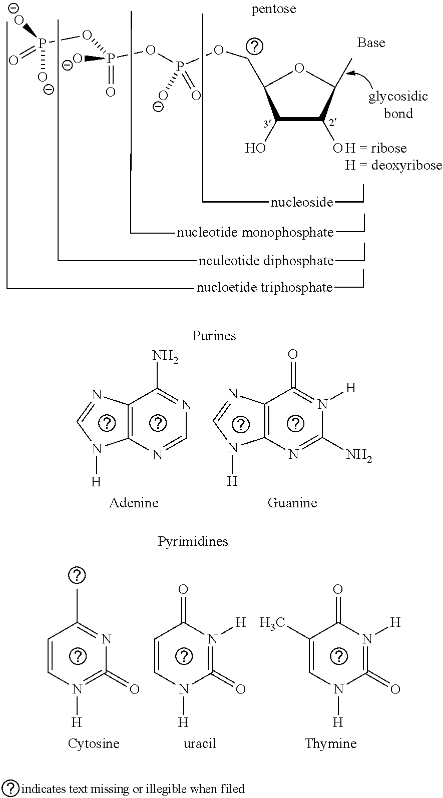

Many enzymes are involved in the hydrolysis of nucleic acids, they can be simply classified as follows:
Nucleases (DNA ases or RNA ases) act on phosphate bonds in nucleic acids; according to their mode of action (endo or exo), nucleases release oligonuclotides or a nucleotide+a residual (n−1) oligonucleotide.
Nucleotidases hydrolyze nucleotide into nucleoside, releasing a phosphate group.
Nucleosidases are N-glycosylases which hydrolyze nucleosides into nucleic bases and ribose or deoxyribose.
Some N-Glucosidases can also act on nucleotides to release nucleic bases and phosphorylated ribose or phosphorylated deoxyribose.
These phosphorylated pentoses can be hydrolyzed by phosphatases to release the free sugar plus a phosphate group.

Apart phosphodiesterase, the other malt sprouts enzymes involved in the pathway of total hydrolysis of nucleic acids are very poorly known.

In a particular embodiment, a composition consisting of malt sprouts fraction extract wherein the malt sprouts fraction extract comprises at least 0.5%, and less than 98% of dry matter by total weight of the malt sprouts fraction extract, is characterized at least by:
Total Nitrogen content from 40 to 80 g/kg of dry matter
Free Amino Nitrogen content from 6 to 30 g/kg of dry matter, and
Nucleic Acid Derivatives such as purines and pyrimidines bases in an amount from 4 to 25 g/kg of dry matter.
PUPY content from 3 to 20 g/kg of dry matter
Ratio PUPY/Nucleic Acid Derivatives higher than 80%.

The above-mentioned composition according to the invention is characterized by a ratio PUPY/Nucleic Acid Derivatives from 80 to 100%.

In the sense of the present invention, a composition consisting of a malt sprouts fraction extract and a malt sprouts fraction extract are equivalent and these terms can be used interchangeably.

In a particular embodiment, a composition according to the invention, consisting of malt sprouts fraction extract, wherein the extract is in a liquid form, preferably a diluted juice, comprising at least 0.5%, and less than 1.1% of dry matter by total weight of the malt sprouts fraction extract, is characterized at least by:
Total Nitrogen content from 40 to 80 g/kg of dry matter
Free Amino Nitrogen content from 6 to 30 g/kg of dry matter, and
Nucleic Acid Derivatives such as purines and pyrimidines bases in an amount from 5 to 25 g/kg of dry matter.
PUPY content from 4 to 20 g/kg of dry matter
Ratio PUPY/Nucleic Acid Derivatives higher than 80%.

The above-mentioned composition according to the invention is characterized by a ratio PUPY/Nucleic Acid Derivatives from 80 to 100%.

In a particular embodiment, a composition according to the invention, consisting of malt sprouts fraction extract, wherein the extract is in a liquid form, preferably a diluted juice, comprising at least 1.1%, and less than 3.5% of dry matter by total weight of the malt sprouts fraction extract, is characterized at least by:
Total Nitrogen content from 40 to 80 g/kg of dry matter
Free Amino Nitrogen content from 6 to 30 g/kg of dry matter, and
Nucleic Acid Derivatives such as purines and pyrimidines bases in an amount from 5 to 25 g/kg of dry matter.
PUPY content from 4 to 20 g/kg of dry matter
Ratio PUPY/Nucleic Acid Derivatives higher than 80%.

The above-mentioned composition according to the invention is characterized by a ratio PUPY/Nucleic Acid Derivatives from 80 to 100%.

In a particular embodiment, a composition according to the invention, consisting of malt sprouts fraction extract, wherein the extract is in a liquid form, preferably a diluted juice, comprising at least 3.5%, and less than 5% of dry matter by total weight of the malt sprouts fraction extract, is characterized at least by:
Total Nitrogen content from 40 to 80 g/kg of dry matter
Free Amino Nitrogen content from 6 to 30 g/kg of dry matter, and
Nucleic Acid Derivatives such as purines and pyrimidines bases in an amount from 5 to 25 g/kg of dry matter.
PUPY content from 4 to 20 g/kg of dry matter
Ratio PUPY/Nucleic Acid Derivatives higher than 80%.

The above-mentioned composition according to the invention is characterized by a ratio PUPY/Nucleic Acid Derivatives from 80 to 100%.

In a particular embodiment a composition according to the invention, consisting of malt sprouts fraction extract, wherein the extract is in a liquid form, preferably a concentrated juice, comprising at least 5%, and less than 7% of dry matter by total weight of the malt sprouts fraction extract, is characterized at least by:
Total Nitrogen content from 40 to 80 g/kg of dry matter
Free Amino Nitrogen content from 6 to 30 g/kg of dry matter, and
Nucleic Acid Derivatives such as purines and pyrimidines bases in an amount from 5 to 25 g/kg of dry matter.
PUPY content from 4 to 20 g/kg of dry matter
Ratio PUPY/Nucleic Acid Derivatives higher than 80%.

The above-mentioned composition according to the invention is characterized by a ratio PUPY/Nucleic Acid Derivatives from 80 to 100%.

In a particular embodiment, a composition according to the invention, consisting of malt sprouts fraction extract, wherein the extract is in a liquid form, preferably a diluted juice, comprising at least 0.5%, and less than 1.1%, or at least 1.1%, and less than 3.5%, or at least 3.5%, and less than 5%, or at least 5%, and less than 7% of dry matter by total weight of the malt sprouts fraction extract, is characterized at least by:
Total Nitrogen content from 40 to 80 g/kg of dry matter
Free Amino Nitrogen content from 6 to 30 g/kg of dry matter, and
Nucleic Acid Derivatives such as purines and pyrimidines bases in an amount from 5 to 25 g/kg of dry matter.
PUPY content from 4 to 20 g/kg of dry matter
Ratio PUPY/Nucleic Acid Derivatives higher than 80%.

The above-mentioned composition according to the invention is characterized by a ratio PUPY/Nucleic Acid Derivatives from 80 to 100%.

In a particular embodiment, a composition according to the invention, consisting of malt sprouts fraction extract, wherein the extract is in a liquid form, preferably a concentrated juice, comprising at least 7%, and less than 10% of dry matter by total weight of the malt sprouts fraction extract, is characterized at least by:
Total Nitrogen content from 40 to 80 g/kg of dry matter
Free Amino Nitrogen content from 6 to 30 g/kg of dry matter, and
Nucleic Acid Derivatives such as purines and pyrimidines bases in an amount from 4 to 25 g/kg of dry matter.
PUPY content from 3 to 20 g/kg of dry matter
Ratio PUPY/Nucleic Acid Derivatives higher than 80%.

The above-mentioned composition according to the invention is characterized by a ratio PUPY/Nucleic Acid Derivatives from 80 to 100%.

In a particular embodiment, a composition according to the invention, consisting of malt sprouts fraction extract, wherein the extract is in a liquid form, preferably a concentrated juice, comprising at least 10%, and less than 15% of dry matter by total weight of the malt sprouts fraction extract, is characterized at least by:
Total Nitrogen content from 40 to 80 g/kg of dry matter
Free Amino Nitrogen content from 6 to 30 g/kg of dry matter, and
Nucleic Acid Derivatives such as purines and pyrimidines bases in an amount from 4 to 25 g/kg of dry matter.
PUPY content from 3 to 20 g/kg of dry matter
Ratio PUPY/Nucleic Acid Derivatives higher than 80%.

The above-mentioned composition according to the invention is characterized by a ratio PUPY/Nucleic Acid Derivatives from 80 to 100%.

In a particular embodiment, a composition according to the invention, consisting of malt sprouts fraction extract, wherein the extract is in a liquid form, preferably a concentrated juice, comprising at least 7%, and less than 10%, or at least 10%, and less than 15% of dry matter by total weight of the malt sprouts fraction extract and characterized at least by:
Total Nitrogen content from 40 to 80 g/kg of dry matter
Free Amino Nitrogen content from 6 to 30 g/kg of dry matter, and
Nucleic Acid Derivatives such as purines and pyrimidines bases in an amount from 4 to 25 g/kg of dry matter.
PUPY content from 3 to 20 g/kg of dry matter
Ratio PUPY/Nucleic Acid Derivatives higher than 80%.

The above-mentioned composition according to the invention is characterized by a ratio PUPY/Nucleic Acid Derivatives from 80 to 100%.

In a particular embodiment, a composition according to the invention, consisting of malt sprouts fraction extract, wherein the extract is in a liquid form or a solid form, preferably a concentrated juice, a slurry or a paste, comprising at least 15%, and less than 60% of dry matter by total weight of the malt sprouts fraction extract, is characterized at least by:
Total Nitrogen content from 40 to 80 g/kg of dry matter
Free Amino Nitrogen content from 6 to 30 g/kg of dry matter, and
Nucleic Acid Derivatives such as purines and pyrimidines bases in an amount from 4 to 25 g/kg of dry matter.
PUPY content from 3 to 20 g/kg of dry matter
Ratio PUPY/Nucleic Acid Derivatives higher than 80%.

The above-mentioned composition according to the invention is characterized by a ratio PUPY/Nucleic Acid Derivatives from 80 to 100%.

In a particular embodiment, a composition according to the invention, consisting of malt sprouts fraction extract, wherein the extract is in a solid form, preferably a paste or a powder, comprising at least 60%, and less than 98% of dry matter by total weight of the malt sprouts fraction extract, is characterized at least by:
Total Nitrogen content from 40 to 80 g/kg of dry matter
Free Amino Nitrogen content from 6 to 30 g/kg of dry matter, and
Nucleic Acid Derivatives such as purines and pyrimidines bases in an amount from 4 to 25 g/kg of dry matter.
PUPY content from 3 to 20 g/kg of dry matter
Ratio PUPY/Nucleic Acid Derivatives higher than 80%.

The above-mentioned composition according to the invention is characterized by a ratio PUPY/Nucleic Acid Derivatives from 80 to 100%.

In a particular embodiment, in a composition consisting of malt sprouts fraction extract according to the invention, the malt is selected from barley, wheat, rye, spelt, corn, millet sorghum, oat, triticale, rice and mixtures thereof, preferably barley.

In a particular embodiment, a composition consisting of malt sprouts fraction extract according to the invention, is obtained from an aqueous extraction of malt sprouts fractions obtained by a sieving process and comprising a content of rootlets and acrospires higher than 50%, preferably higher than 60%, more preferably higher than 65% and even more preferably between 65% and 85% by total weight of fractions.

According to the invention, the term “sieving process” refers to techniques used to separate particles from a mixture based on the difference in size of particles. It uses sieve plates for separation of coarse particles from finer particles.

In a particular embodiment, a composition consisting of malt sprouts fraction extract according to the invention, wherein the malt is selected from barley, wheat, rye, spelt, corn, millet sorghum, oat, triticale, rice and mixtures thereof, preferably barley, is obtained from an aqueous extraction of malt sprouts fractions obtained by a sieving process and comprising a content of rootlets and acrospires higher than 50%, preferably higher than 60%, more preferably higher than 65% and even more preferably between 65% and 85% by total weight of fractions.

In a particular embodiment, a composition consisting of malt sprouts fraction extract according to the invention, is obtained from an aqueous extraction of malt sprouts fractions characterized at least by:
Dry matter by total weight of the malt sprouts from 92% to 98%,
Crude protein content from 22.5 to 37 g/100 g of malt sprout fraction
Free Amino Nitrogen content higher than 200 mg/100 g of dry matter.

In a particular embodiment, a composition consisting of malt sprouts fraction extract according to the invention, is obtained from an aqueous extraction of malt sprouts fractions characterized at least by:
Dry matter by total weight of the malt sprouts from 92% to 98%,
Crude protein content from 22.5 to 37 g/100 g of malt sprout fraction
Free Amino Nitrogen content higher than 200 mg/100 g of dry matter,
and preferably containing:
Total fibers content from 43 to 52 g/100 g of dry matter,
Total carbohydrates content from 7 to 12 g/100 g of dry matter,
Total sugars content from 4 to 6 g/100 g of dry matter,
Fats content below 1 g/100 g
Ash content from 5 to 7 g/100 g.

In a particular embodiment, a composition consisting of malt sprouts fraction extract according to the invention, is obtained from an aqueous extraction of malt sprouts fractions characterized at least by:
Dry matter by total weight of the malt sprouts from 92% to 98%,
Crude protein content from 22.5 to 37 g/100 g of dry matter,
Free Amino Nitrogen content higher than 200 mg/100 g of dry matter,
Total fibers content from 43 to 52 g/100 g of dry matter,
Total carbohydrates content from 7 to 12 g/100 g of dry matter,
Total sugars content from 4 to 6 g/100 g of dry matter,
Fats content below 1 g/100 g
Ash content from 5 to 7 g/100 g.

In a particular embodiment, a composition consisting of malt sprouts fraction extract according to the invention, is obtained from an aqueous extraction of malt sprouts fractions of barley obtained by a sieving process and comprising a content of rootlets and acrospires higher than 50%, preferably higher than 60%, more preferably higher than 65% and even more preferably between 65 and 85% by total weight of fractions.

Another object of the invention is a process of preparation of malt sprouts fraction extract comprising the following steps:
a. Diffusion of diluted malt sprouts fractions in aqueous phase, optionally followed by a finishing step at pH<6 or >8 and a thermal treatment at 100° C. preferably for 20 minutes, to obtain two phases, an aqueous one and a solid one;
   or hydrolysis of diluted malt sprouts fractions in the presence of exogenous enzymes, optionally followed by a finishing step at pH<6 or >8 and thermal treatment at 100° C. preferably for 20 minutes, to obtain two phases, an aqueous one and a solid one;
b. Separation of the aqueous phase from the solid phase, preferably by centrifugation, to recover the aqueous phase;
c. Optionally microfiltration of the aqueous phase on plate filters to remove fine insoluble particles with a lower size than 1 μm, preferably lower than 0.8 μm and more preferably from 0.2 μm to 0.8 μm;
d. Optionally, treatment of the aqueous phase obtained in step (b) or (c) in order to obtain the desired % of dry matter by total weight of malt sprouts fraction extract;
e. And optionally a sterilization step.

Advantageously, the diffusion is carried out at a temperature of 20° C. to 50° C.

Advantageously, the hydrolysis is carried out at a temperature of 20° C. to 70° C. depending on the enzyme which is used.

Advantageously, the pH of hydrolysis should be adapted to the optimum pH of the enzymatic cocktail which is used.

The aim of the Inventors was to hydrolyze nucleic acids into nucleosides and bases (purines and pyrimidines) in order to maximize the proportion of bases. Such hydrolysis requires particular conditions because of the complexity of occurring reactions which involve endogenous nucleases from malt rootlets and acrospires, associated or not to exogenous enzymes (including exogenous nucleases selected in the classification defined on page 7). This step is associated to a proteolysis, either moderated by the endogenous proteases from malt rootlets and acrospires, or amplified by exogenous proteases.

After the diffusion step (with or without exogenous enzymes), obtained mixtures are alkalinized by adjusting the pH≥8 or acidified at pH≤6 and then heated at temperature around 100° C. during few minutes (up to 20 minutes), this step is defined as the finishing step according to the invention. This step deactivates endogenous enzymes from rootlets and acrospires, including nucleases, and favors the alkaline hydrolysis in order to maximize the obtention of nucleic acids derivatives, i.e. nucleotides, nucleosides and purine and pyrimidine bases. Time, temperature and pH of the finishing step could be adjusted depending on the nucleic bases concentration desired.

These operations allow the improvement of extraction rates and the adjustment of the extracts composition in nitrogen derivatives (Total Nitrogen content, Free Amino Nitrogen, Nucleic acid derivatives including purine and pyrimidine bases).

According to the invention, the term “treatment of the aqueous phase” refers to concentration treatments used to increase dry matter of the aqueous extract by means of ultrafiltration, reverse osmosis, evaporation, spray-drying, freeze-drying and other concentrating, evaporating or drying systems.

According to the invention, the term “sterilization step” refers to the destruction and/or elimination of all the micro-organisms from a matrix, by means of heating, addition of antiseptics, microfiltration, or irradiation, in order to avoid the transmission of pathogens and the development of alterations.

In the sense of the invention, the “optimized diffusion process” refers to a process wherein the malt sprouts fractions are mixed and diffused in water, with an additional finishing step as further disclosed.

In the sense of the invention, the “simple hydrolysis process” refers to a process wherein the malt sprouts fractions are mixed in water and submitted to a hydrolysis in the presence of enzymes as disclosed herein, without a finishing step.

In the sense of the invention, the “optimized hydrolysis process” refers to a process wherein the malt sprouts fractions are mixed in water and submitted to a hydrolysis in the presence of enzymes as disclosed herein, with a finishing step as further disclosed.

The terms “diffusion process” and “diffusion method” are used interchangeably. And the terms “hydrolysis process” and “hydrolysis method” are used interchangeably.

Another object of the invention is a process of preparation of a protein mixture containing malt sprouts fraction extract and additional nitrogen compounds, comprising the following steps:
a. Diffusion of diluted malt sprouts fractions and primary protein sources, in aqueous phase, optionally followed by a finishing step at pH<6 or >8 and a thermal treatment at 100° C. preferably for 20 minutes, to obtain two phases, an aqueous one and a solid one;
   or hydrolysis of diluted malt sprouts fractions and primary protein sources, in the presence of exogenous enzymes, optionally followed by a finishing step at pH<6 or >8 and thermal treatment at 100° C. preferably for 20 minutes, to obtain two phases, an aqueous one and a solid one;
b. Separation of the aqueous phase from the solid phase, preferably by centrifugation, to recover the aqueous phase;
c. Optionally microfiltration of the aqueous phase on plate filters to remove fine insoluble particles with a lower size than 1 μm, preferably lower than 0.8 μm and more preferably from 0.2 μm to 0.8 μm;
d. Optionally, treatment of the aqueous phase obtained in step (b) or (c) in order to obtain the desired % of dry matter by total weight of malt sprouts fraction extract;
e. And optionally a sterilization step.

Advantageously, in the above-mentioned process, the diffusion is carried out at a temperature of 20° C. to 50° C.

Advantageously, in the above-mentioned process, the hydrolysis is carried out at a temperature of 20° C. to 70° C., depending on the enzyme which is used.

This embodiment allows to enrich malt sprouts fraction extract in nitrogen compounds.

According to the invention, the term “additional nitrogen compounds” refers to a mixture of proteins, peptides, amino acids, DAN, nitrogen in the form of ammonia (NH3-N) and ammonium (NH4+−N) coming from the hydrolysis of primary protein sources.

In the sense of the invention, the term “primary protein source” refers to protein matter originating from various vegetal, animal, or microorganism, such as soy, rapeseed, pea, alfalfa, wheat protein, casein, gelatin, meat, or yeast.

Such primary protein sources can be found in the forms of meal, flour, concentrate or isolate.

The normal protein content in standard “malt sprouts” is in the range from 18 to 22%. Among the main components (rootlets, acrospires and husks), rootlets and acrospires contain respectively about 32% and 30% of proteins. Malt sprouts fraction extracts according to the invention can be obtained from separated protein-rich components or from fractions containing a high proportion of these components. It is also possible to use dry milling techniques such as micronisation and turbo separation to obtain protein-enriched malt sprouts fractions.

However, it can be useful to combine malt sprouts fractions with other primary protein sources to obtain extracts with higher contents of nitrogen (for instance >60 g N/kg dm) and FAN (for instance >15 g/kg dm) and lower ratios of PUPY/FAN than malt sprouts fraction extracts alone.

Primary protein sources can be mixed with malt sprout fractions for treating the resulting mixture according to the extraction processes of the invention. The primary protein source is characterized by the protein content which can be relatively low, (from about 20% to about 45%). For instance: brewers or distillers spent grains DDGS (Dried Distillers Grains with Solubles), rapeseed meals, soy meal . . . . Sources with medium protein content (about 50 to about 75%) are mostly concentrated from the same sources or extraction products like wheat gluten. Sources with high protein content (above about 75%) are mostly isolated from the same sources or meats, fish flesh, or extracted proteins such as casein, gelatin . . . .

However, the process of preparation of a protein mixture containing malt sprouts fraction extract and additional nitrogen compounds is preferably applied to selected primary protein sources, to obtain extraction products with defined characteristics (for instance combining an increased total nitrogen content and a specific ratio of PUPY/FAN). Selected primary protein sources with sufficiently high protein content, combining high soluble yield and high protein yield are preferred. The possible proportion of such primary protein sources to malt sprout fractions is respectively in the range 10/90 to 80/20 (weight protein source/weight malt sprout fraction), in particular 10/90, 20/80, 30/70, 40/60, 50/50, 60/40, 70/30, 80/20 and preferably in the range 50/50 to 20/80.

For example, the process of preparation of a protein mixture containing malt sprouts fraction extract and additional nitrogen compounds comprising 80 parts of malt sprouts fraction (30% protein) and 20 parts of a primary protein source (76%) allows to yield an extract containing 8.9% total nitrogen (55.6% crude protein), 20.1 g FAN/kg d.m. and 6.07 g PUPY/kg d.m. When changing the proportion to 50/50, the mixture of malt sprouts fraction extract and additional nitrogen compounds contains 10.9% total nitrogen (68.1% crude protein), 25.4 g FAN/kg d.m. and 3.55 g PUPY/kg d.m. These examples show that the characteristics of the mixture of malt sprouts fraction extract and additional nitrogen compounds can be tuned to optimize the relative proportions of the nitrogen components.

In a particular embodiment, the process of preparation of malt sprouts fraction extract according to the invention comprises the following steps:
a. Diffusion of diluted malt sprouts fractions in aqueous phase, followed by a finishing step at pH<6 or >8 and a thermal treatment at 100° C. preferably for 20 minutes, to obtain two phases, an aqueous one and a solid one;
b. Separation of the aqueous phase from the solid phase, preferably by centrifugation, to recover the aqueous phase;
c. Optionally microfiltration of the aqueous phase on plate filters to remove fine insoluble particles with a lower size than 1 μm, preferably lower than 0.8 μm and more preferably from 0.2 μm to 0.8 μm;
d. Optionally, treatment of the aqueous phase obtained in step (b) or (c) in order to obtain the desired % of dry matter by total weight of malt sprouts fraction extract;
e. And optionally a sterilization step.

Advantageously, in the above-mentioned process, the diffusion is carried out at a temperature of 20° C. to 50° C.

In a particular embodiment, the process of preparation of malt sprouts fraction extract according to the invention comprises the following steps:
a. Hydrolysis of diluted malt sprouts fractions in the presence of exogenous enzymes, to obtain two phases, an aqueous one and a solid one;
b. Separation of the aqueous phase from the solid phase, preferably by centrifugation, to recover the aqueous phase;
c. Optionally microfiltration of the aqueous phase on plate filters to remove fine insoluble particles with a lower size than 1 μm, preferably lower than 0.8 μm and more preferably from 0.2 μm to 0.8 μm;
d. Optionally, treatment of the aqueous phase obtained in step (b) or (c) in order to obtain the desired % of dry matter by total weight of malt sprouts fraction extract;
e. And optionally a sterilization step.

Advantageously, in the above-mentioned process, the hydrolysis is carried out at a temperature of 20° C. to 70° C. depending on the enzyme which is used.

In a particular embodiment, the process of preparation of malt sprouts fraction extract according to the invention comprises the following steps:
a. Hydrolysis of diluted malt sprouts fractions in the presence of exogenous enzymes, followed by a finishing step at pH<6 or >8 and thermal treatment at 100° C. preferably for 20 minutes, to obtain two phases, an aqueous one and a solid one;
b. Separation of the aqueous phase from the solid phase, preferably by centrifugation, to recover the aqueous phase;
c. Optionally microfiltration of the aqueous phase on plate filters to remove fine insoluble particles with a lower size than 1 μm, preferably lower than 0.8 μm and more preferably from 0.2 μm to 0.8 μm;
d. Optionally, treatment of the aqueous phase obtained in step (b) or (c) in order to obtain the desired % of dry matter by total weight of malt sprouts fraction extract;
e. And optionally a sterilization step.

Advantageously, in the above-mentioned process, the hydrolysis is carried out at a temperature of 20° C. to 70° C., depending on the enzyme which is used.

Another object of the invention is a process of preparation of protein mixture containing malt sprouts fraction extract according to the invention and additional nitrogen compounds comprising the following steps:
a. Hydrolysis of diluted malt sprouts fractions and primary protein source, in the presence of exogenous enzymes, followed by a finishing step at pH<6 or >8 and thermal treatment at 100° C. preferably for 20 minutes, to obtain two phases, an aqueous one and a solid one;
b. Separation of the aqueous phase from the solid phase, preferably by centrifugation, to recover the aqueous phase;
c. Optionally microfiltration of the aqueous phase on plate filters to remove fine insoluble particles with a lower size than 1 μm, preferably lower than 0.8 μm and more preferably from 0.2 μm to 0.8 μm;
d. Optionally, treatment of the aqueous phase obtained in step (b) or (c) in order to obtain the desired % of dry matter by total weight of malt sprouts fraction extract;
e. And optionally a sterilization step.

Advantageously, in the above-mentioned process, the hydrolysis is carried out at a temperature of 20° C. to 70° C., depending on the enzyme which is used.

In a particular embodiment, the process of preparation of malt sprouts fraction extract according to the invention and wherein the malt sprouts fraction extract comprises at least 0.5%, and less than 1.1% of dry matter by total weight of the malt sprouts fraction extract, comprises the following steps:
a. Diffusion of diluted malt sprouts fractions in aqueous phase followed by a finishing step at pH<6 or >8 and a thermal treatment at 100° C. preferably for 20 minutes, to obtain two phases, an aqueous one and a solid one;
b. Separation of the aqueous phase from the solid phase, preferably by centrifugation, to recover the aqueous phase;
c. Optionally microfiltration of the aqueous phase on plate filters to remove fine insoluble particles with a lower size than 1 μm, preferably lower than 0.8 μm and more preferably from 0.2 μm to 0.8 μm;
d. Optionally, treatment of the aqueous phase obtained in step (b) or (c) in order to obtain a % of dry matter from 0.5 to 1.1% by total weight of malt sprouts fraction extract;
e. And optionally a sterilization step.

In a particular embodiment, the process of preparation of malt sprouts fraction extract according to the invention and wherein the malt sprouts fraction extract comprises at least 1.1%, and less than 3.5% of dry matter by total weight of the malt sprouts fraction extract, comprises the following steps:
a. Diffusion of diluted malt sprouts fractions in aqueous phase followed by a finishing step at pH<6 or >8 and a thermal treatment at 100° C. preferably for 20 minutes, to obtain two phases, an aqueous one and a solid one;
b. Separation of the aqueous phase from the solid phase, preferably by centrifugation, to recover the aqueous phase;
c. Optionally microfiltration of the aqueous phase on plate filters to remove fine insoluble particles with a lower size than 1 μm, preferably lower than 0.8 μm and more preferably from 0.2 μm to 0.8 μm;
d. Optionally, treatment of the aqueous phase obtained in step (b) or (c) in order to obtain a % of dry matter from 1.1 to 3.5% by total weight of malt sprouts fraction extract;
e. And optionally a sterilization step.

In a particular embodiment, the process of preparation of malt sprouts fraction extract according to the invention and wherein the malt sprouts fraction extract comprises at least 3.5%, and less than 5% of dry matter by total weight of the malt sprouts fraction extract, comprises the following steps:
a. Diffusion of diluted malt sprouts fractions in aqueous phase followed by a finishing step at pH<6 or >8 and a thermal treatment at 100° C. preferably for 20 minutes, to obtain two phases, an aqueous one and a solid one;
b. Separation of the aqueous phase from the solid phase, preferably by centrifugation, to recover the aqueous phase;
c. Optionally microfiltration of the aqueous phase on plate filters to remove fine insoluble particles with a lower size than 1 μm, preferably lower than 0.8 μm and more preferably from 0.2 μm to 0.8 μm;
d. Optionally, treatment of the aqueous phase obtained in step (b) or (c) in order to obtain a % of dry matter from 3.5 to 5% by total weight of malt sprouts fraction extract;
e. And optionally a sterilization step.

In a particular embodiment, the process of preparation of malt sprouts fraction extract according to the invention and wherein the malt sprouts fraction extract comprises at least 5%, and less than 7% of dry matter by total weight of the malt sprouts fraction extract, comprises the following steps:
a. Diffusion of diluted malt sprouts fractions in aqueous phase followed by a finishing step at pH<6 or >8 and a thermal treatment at 100° C. preferably for 20 minutes, to obtain two phases, an aqueous one and a solid one;
b. Separation of the aqueous phase from the solid phase, preferably by centrifugation, to recover the aqueous phase;
c. Optionally microfiltration of the aqueous phase on plate filters to remove fine insoluble particles with a lower size than 1 μm, preferably lower than 0.8 μm and more preferably from 0.2 μm to 0.8 μm;
d. Optionally, treatment of the aqueous phase obtained in step (b) or (c) in order to obtain a % of dry matter from 5 to 7% by total weight of malt sprouts fraction extract;
e. And optionally a sterilization step.

In a particular embodiment, the process of preparation of malt sprouts fraction extract according to the invention and wherein the malt sprouts fraction extract comprises at least 7%, and less than 10% of dry matter by total weight of the malt sprouts fraction extract, comprises the following steps:
a. Diffusion of diluted malt sprouts fractions in aqueous phase followed by a finishing step at pH<6 or >8 and a thermal treatment at 100° C. preferably for 20 minutes, to obtain two phases, an aqueous one and a solid one;
b. Separation of the aqueous phase from the solid phase, preferably by centrifugation, to recover the aqueous phase;
c. Optionally microfiltration of the aqueous phase on plate filters to remove fine insoluble particles with a lower size than 1 μm, preferably lower than 0.8 μm and more preferably from 0.2 μm to 0.8 μm;
d. Optionally, treatment of the aqueous phase obtained in step (b) or (c) in order to obtain a % of dry matter from 7 to 10% by total weight of malt sprouts fraction extract;
e. And optionally a sterilization step.

In a particular embodiment, the process of preparation of malt sprouts fraction extract according to the invention and wherein the malt sprouts fraction extract comprises at least 10%, and less than 15% of dry matter by total weight of the malt sprouts fraction extract, comprises the following steps:
a. Diffusion of diluted malt sprouts fractions in aqueous phase followed by a finishing step at pH<6 or >8 and a thermal treatment at 100° C. preferably for 20 minutes, to obtain two phases, an aqueous one and a solid one;
b. Separation of the aqueous phase from the solid phase, preferably by centrifugation, to recover the aqueous phase;
c. Optionally microfiltration of the aqueous phase on plate filters to remove fine insoluble particles with a lower size than 1 μm, preferably lower than 0.8 μm and more preferably from 0.2 μm to 0.8 μm;
d. Optionally, treatment of the aqueous phase obtained in step (b) or (c) in order to obtain a % of dry matter from 10 to 15% by total weight of malt sprouts fraction extract;
e. And optionally a sterilization step.

In a particular embodiment, the process of preparation of malt sprouts fraction extract according to the invention and wherein the malt sprouts fraction extract comprises at least 15%, and less than 60% of dry matter by total weight of the malt sprouts fraction extract, comprises the following steps:
a. Diffusion of diluted malt sprouts fractions in aqueous phase followed by a finishing step at pH<6 or >8 and a thermal treatment at 100° C. preferably for 20 minutes, to obtain two phases, an aqueous one and a solid one;
b. Separation of the aqueous phase from the solid phase, preferably by centrifugation, to recover the aqueous phase;
c. Optionally microfiltration of the aqueous phase on plate filters to remove fine insoluble particles with a lower size than 1 μm, preferably lower than 0.8 μm and more preferably from 0.2 μm to 0.8 μm;
d. Treatment of the aqueous phase obtained in step (b) or (c) in order to obtain a % of dry matter from 15 to 60% by total weight of malt sprouts fraction extract;
e. And optionally a sterilization step.

In a particular embodiment, the process of preparation of malt sprouts fraction extract according to the invention and wherein the malt sprouts fraction extract comprises at least 60%, and less than 98% of dry matter by total weight of the malt sprouts fraction extract, comprises the following steps:
a. Diffusion of diluted malt sprouts fractions in aqueous phase followed by a finishing step at pH<6 or >8 and a thermal treatment at 100° C. preferably for 20 minutes, to obtain two phases, an aqueous one and a solid one;
b. Separation of the aqueous phase from the solid phase, preferably by centrifugation, to recover the aqueous phase;
c. Optionally microfiltration of the aqueous phase on plate filters to remove fine insoluble particles with a lower size than 1 μm, preferably lower than 0.8 μm and more preferably from 0.2 μm to 0.8 μm;
d. Treatment of the aqueous phase obtained in step (b) or (c) in order to obtain a % of dry matter from 60 to 98% by total weight of malt sprouts fraction extract;
e. And optionally a sterilization step.

In a particular embodiment, the process of preparation of malt sprouts fraction extract according to the invention and wherein the malt sprouts fraction extract comprises at least 7%, and less than 10% of dry matter by total weight of the malt sprouts fraction extract, comprises the following steps:
a. Hydrolysis of diluted malt sprouts fractions in the presence of exogenous enzymes, to obtain two phases, an aqueous one and a solid one;
b. Separation of the aqueous phase from the solid phase, preferably by centrifugation, to recover the aqueous phase;
c. Optionally microfiltration of the aqueous phase on plate filters to remove fine insoluble particles with a lower size than 1 μm, preferably lower than 0.8 μm and more preferably from 0.2 μm to 0.8 μm;
d. Optionally, treatment of the aqueous phase obtained in step (b) or (c) in order to obtain a % of dry matter from 7 to 10% by total weight of malt sprouts fraction extract;
e. And optionally a sterilization step.

In a particular embodiment, the process of preparation of malt sprouts fraction extract according to the invention and wherein the malt sprouts fraction extract comprises at least 10%, and less than 15% of dry matter by total weight of the malt sprouts fraction extract, comprises the following steps:
a. Hydrolysis of diluted malt sprouts fractions in the presence of exogenous enzymes, to obtain two phases, an aqueous one and a solid one;
b. Separation of the aqueous phase from the solid phase, preferably by centrifugation, to recover the aqueous phase;
c. Optionally microfiltration of the aqueous phase on plate filters to remove fine insoluble particles with a lower size than 1 μm, preferably lower than 0.8 μm and more preferably from 0.2 μm to 0.8 μm;
d. Optionally, treatment of the aqueous phase obtained in step (b) or (c) in order to obtain a % of dry matter from 10 to 15% by total weight of malt sprouts fraction extract;
e. And optionally a sterilization step.

In a particular embodiment, the process of preparation of malt sprouts fraction extract according to the invention and wherein the malt sprouts fraction extract comprises at least 15%, and less than 60% of dry matter by total weight of the malt sprouts fraction extract, comprises the following steps:
a. Hydrolysis of diluted malt sprouts fractions in the presence of exogenous enzymes, to obtain two phases, an aqueous one and a solid one;
b. Separation of the aqueous phase from the solid phase, preferably by centrifugation, to recover the aqueous phase;
c. Optionally microfiltration of the aqueous phase on plate filters to remove fine insoluble particles with a lower size than 1 μm, preferably lower than 0.8 μm and more preferably from 0.2 μm to 0.8 μm;
d. Treatment of the aqueous phase obtained in step (b) or (c) in order to obtain a % of dry matter from 15 to 60% by total weight of malt sprouts fraction extract;
e. And optionally a sterilization step.

In a particular embodiment, the process of preparation of malt sprouts fraction extract according to the invention and wherein the malt sprouts fraction extract comprises at least 60%, and less than 98% of dry matter by total weight of the malt sprouts fraction extract, comprises the following steps:
a. Hydrolysis of diluted malt sprouts fractions in the presence of exogenous enzymes, to obtain two phases, an aqueous one and a solid one;
b. Separation of the aqueous phase from the solid phase, preferably by centrifugation, to recover the aqueous phase;
c. Optionally microfiltration of the aqueous phase on plate filters to remove fine insoluble particles with a lower size than 1 μm, preferably lower than 0.8 μm and more preferably from 0.2 μm to 0.8 μm;
d. Treatment of the aqueous phase obtained in step (b) or (c) in order to obtain a % of dry matter from 60 to 98% by total weight of malt sprouts fraction extract;
e. And optionally a sterilization step.

In a particular embodiment, the process of preparation of malt sprouts fraction extract according to the invention and wherein the malt sprouts fraction extract comprises at least 0.5%, and less than 1.1% of dry matter by total weight of the malt sprouts fraction extract, comprises the following steps:
a. Hydrolysis of diluted malt sprouts fractions in the presence of exogenous enzymes, followed by a finishing step at pH<6 or >8 and thermal treatment at 100° C. preferably for 20 minutes, to obtain two phases, an aqueous one and a solid one;
b. Separation of the aqueous phase from the solid phase, preferably by centrifugation, to recover the aqueous phase;
c. Optionally microfiltration of the aqueous phase on plate filters to remove fine insoluble particles with a lower size than 1 μm, preferably lower than 0.8 μm and more preferably from 0.2 μm to 0.8 μm;
d. Optionally, treatment of the aqueous phase obtained in step (b) or (c) in order to obtain a % of dry matter from 0.5 to 1.1% by total weight of malt sprouts fraction extract;
e. And optionally a sterilization step.

In a particular embodiment, the process of preparation of malt sprouts fraction extract according to the invention and wherein the malt sprouts fraction extract comprises at least 1.1%, and less than 3.5% of dry matter by total weight of the malt sprouts fraction extract, comprises the following steps:
a. Hydrolysis of diluted malt sprouts fractions in the presence of exogenous enzymes, followed by a finishing step at pH<6 or >8 and thermal treatment at 100° C. preferably for 20 minutes, to obtain two phases, an aqueous one and a solid one;
b. Separation of the aqueous phase from the solid phase, preferably by centrifugation, to recover the aqueous phase;
c. Optionally microfiltration of the aqueous phase on plate filters to remove fine insoluble particles with a lower size than 1 μm, preferably lower than 0.8 μm and more preferably from 0.2 μm to 0.8 μm;
d. Optionally, treatment of the aqueous phase obtained in step (b) or (c) in order to obtain a % of dry matter from 1.1 to 3.5% by total weight of malt sprouts fraction extract;
e. And optionally a sterilization step.

In a particular embodiment, the process of preparation of malt sprouts fraction extract according to the invention and wherein the malt sprouts fraction extract comprises at least 3.5%, and less than 5% of dry matter by total weight of the malt sprouts fraction extract, comprises the following steps:
f. Hydrolysis of diluted malt sprouts fractions in the presence of exogenous enzymes, followed by a finishing step at pH<6 or >8 and thermal treatment at 100° C. preferably for 20 minutes, to obtain two phases, an aqueous one and a solid one;
g. Separation of the aqueous phase from the solid phase, preferably by centrifugation, to recover the aqueous phase;
h. Optionally microfiltration of the aqueous phase on plate filters to remove fine insoluble particles with a lower size than 1 μm, preferably lower than 0.8 μm and more preferably from 0.2 μm to 0.8 μm;
i. Optionally, treatment of the aqueous phase obtained in step (b) or (c) in order to obtain a % of dry matter from 3.5 to 5% by total weight of malt sprouts fraction extract;
j. And optionally a sterilization step.

In a particular embodiment, the process of preparation of malt sprouts fraction extract according to the invention and wherein the malt sprouts fraction extract comprises at least 5%, and less than 7% of dry matter by total weight of the malt sprouts fraction extract, comprises the following steps:
a. Hydrolysis of diluted malt sprouts fractions in the presence of exogenous enzymes, followed by a finishing step at pH<6 or >8 and thermal treatment at 100° C. preferably for 20 minutes, to obtain two phases, an aqueous one and a solid one;
b. Separation of the aqueous phase from the solid phase, preferably by centrifugation, to recover the aqueous phase;
c. Optionally microfiltration of the aqueous phase on plate filters to remove fine insoluble particles with a lower size than 1 μm, preferably lower than 0.8 μm and more preferably from 0.2 μm to 0.8 μm;
d. Optionally, treatment of the aqueous phase obtained in step (b) or (c) in order to obtain a % of dry matter from 5 to 7% by total weight of malt sprouts fraction extract;
e. And optionally a sterilization step.

In a particular embodiment, the process of preparation of malt sprouts fraction extract according to the invention and wherein the malt sprouts fraction extract comprises at least 7%, and less than 10% of dry matter by total weight of the malt sprouts fraction extract, comprises the following steps:
a. Hydrolysis of diluted malt sprouts fractions in the presence of exogenous enzymes, followed by a finishing step at pH<6 or >8 and thermal treatment at 100° C. preferably for 20 minutes, to obtain two phases, an aqueous one and a solid one;
b. Separation of the aqueous phase from the solid phase, preferably by centrifugation, to recover the aqueous phase;
c. Optionally microfiltration of the aqueous phase on plate filters to remove fine insoluble particles with a lower size than 1 μm, preferably lower than 0.8 μm and more preferably from 0.2 μm to 0.8 μm;
d. Optionally, treatment of the aqueous phase obtained in step (b) or (c) in order to obtain a % of dry matter from 7 to 10% by total weight of malt sprouts fraction extract;
e. And optionally a sterilization step.

In a particular embodiment, the process of preparation of malt sprouts fraction extract according to the invention and wherein the malt sprouts fraction extract comprises at least 10%, and less than 15% of dry matter by total weight of the malt sprouts fraction extract, comprises the following steps:
a. Hydrolysis of diluted malt sprouts fractions in the presence of exogenous enzymes, followed by a finishing step at pH<6 or >8 and thermal treatment at 100° C. preferably for 20 minutes, to obtain two phases, an aqueous one and a solid one;
b. Separation of the aqueous phase from the solid phase, preferably by centrifugation, to recover the aqueous phase;
c. Optionally microfiltration of the aqueous phase on plate filters to remove fine insoluble particles with a lower size than 1 μm, preferably lower than 0.8 μm and more preferably from 0.2 μm to 0.8 μm;
d. Optionally, treatment of the aqueous phase obtained in step (b) or (c) in order to obtain a % of dry matter from 10 to 15% by total weight of malt sprouts fraction extract;
e. And optionally a sterilization step.

In a particular embodiment, the process of preparation of malt sprouts fraction extract according to the invention and wherein the malt sprouts fraction extract comprises at least 15%, and less than 60% of dry matter by total weight of the malt sprouts fraction extract, comprises the following steps:
a. Hydrolysis of diluted malt sprouts fractions in the presence of exogenous enzymes, followed by a finishing step at pH<6 or >8 and thermal treatment at 100° C. preferably for 20 minutes, to obtain two phases, an aqueous one and a solid one;
b. Separation of the aqueous phase from the solid phase, preferably by centrifugation, to recover the aqueous phase;
c. Optionally microfiltration of the aqueous phase on plate filters to remove fine insoluble particles with a lower size than 1 μm, preferably lower than 0.8 μm and more preferably from 0.2 μm to 0.8 μm;
d. Treatment of the aqueous phase obtained in step (b) or (c) in order to obtain a % of dry matter from 15 to 60% by total weight of malt sprouts fraction extract;
e. And optionally a sterilization step.

In a particular embodiment, the process of preparation of malt sprouts fraction extract according to the invention and wherein the malt sprouts fraction extract comprises at least 60%, and less than 98% of dry matter by total weight of the malt sprouts fraction extract, comprises the following steps:
a. Hydrolysis of diluted malt sprouts fractions in the presence of exogenous enzymes, followed by a finishing step at pH<6 or >8 and thermal treatment at 100° C. preferably for 20 minutes, to obtain two phases, an aqueous one and a solid one;
b. Separation of the aqueous phase from the solid phase, preferably by centrifugation, to recover the aqueous phase;
c. Optionally microfiltration of the aqueous phase on plate filters to remove fine insoluble particles with a lower size than 1 μm, preferably lower than 0.8 μm and more preferably from 0.2 μm to 0.8 μm;
d. Treatment of the aqueous phase obtained in step (b) or (c) in order to obtain a % of dry matter from 60 to 98% by total weight of malt sprouts fraction extract;
e. And optionally a sterilization step.

Advantageously, the dilution of isolated malt sprouts fractions in aqueous phase, is carried out preferably within a ratio from 1/4 to 1/15, preferably 1/5, 1/7, 1/10 or 1/12, to obtain diluted malt sprouts fractions.

In a particular embodiment, the process of preparation of malt sprouts fraction extract according to the invention comprises the following steps:
a. Dilution of isolated malt sprouts fractions enriched in anyone of rootlets, acrospires, hulk, dust and mixtures thereof in aqueous phase, preferably within a ratio from 1/4 to 1/15, preferably 1/5, 1/7, 1/10 or 1/12, to obtain diluted malt sprouts fractions;
b. Diffusion of these diluted malt sprouts fractions in aqueous phase, followed by a finishing step at pH<6 or >8 and a thermal treatment at 100° C. preferably for 20 minutes, to obtain two phases, an aqueous one and a solid one;
c. Separation of the aqueous phase from the solid phase, preferably by centrifugation, to recover the aqueous phase;
d. Optionally microfiltration of the aqueous phase on plate filters to remove fine insoluble particles with a lower size than 1 μm, preferably lower than 0.8 μm and more preferably from 0.2 μm to 0.8 μm;
e. Optionally, treatment of the aqueous phase obtained in step (c) or (d) in order to obtain the desired % of dry matter by total weight of malt sprouts fraction extract;
f. And optionally a sterilization step.

Advantageously, in the above-mentioned process, the diffusion is carried out at a temperature of 20° C. to 50° C.

In a particular embodiment, the process of preparation of malt sprouts fraction extract according to the invention comprises the following steps:
a. Dilution of isolated malt sprouts fractions enriched in anyone of rootlets, acrospires, hulk, dust and mixtures thereof in aqueous phase, preferably within a ratio from 1/4 to 1/15, preferably 1/5, 1/7, 1/10 or 1/12, to obtain diluted malt sprouts fractions;
b. Hydrolysis of these diluted malt sprouts fractions in the presence of exogenous enzymes, to obtain two phases, an aqueous one and a solid one;
c. Separation of the aqueous phase from the solid phase, preferably by centrifugation, to recover the aqueous phase;
d. Optionally microfiltration of the aqueous phase on plate filters to remove fine insoluble particles with a lower size than 1 μm, preferably lower than 0.8 μm and more preferably from 0.2 μm to 0.8 μm;
e. Optionally, treatment of the aqueous phase obtained in step (c) or (d) in order to obtain the desired % of dry matter by total weight of malt sprouts fraction extract;
f. And optionally a sterilization step.

Advantageously, in the above-mentioned process, the hydrolysis is carried out at a temperature of 20° C. to 70° C., depending on the enzyme which is used.

In a particular embodiment, the process of preparation of malt sprouts fraction extract according to the invention comprises the following steps:
a. Dilution of isolated malt sprouts fractions enriched in anyone of rootlets, acrospires, hulk, dust and mixtures thereof in aqueous phase, preferably within a ratio from 1/4 to 1/15, preferably 1/5, 1/7, 1/10 or 1/12, to obtain diluted malt sprouts fractions;
b. Hydrolysis of these diluted malt sprouts fractions in the presence of exogenous enzymes, followed by a finishing step at pH<6 or >8 and thermal treatment at 100° C. preferably for 20 minutes, to obtain two phases, an aqueous one and a solid one;
c. Separation of the aqueous phase from the solid phase, preferably by centrifugation, to recover the aqueous phase;
d. Optionally microfiltration of the aqueous phase on plate filters to remove fine insoluble particles with a lower size than 1 μm, preferably lower than 0.8 μm and more preferably from 0.2 μm to 0.8 μm;
e. Optionally, treatment of the aqueous phase obtained in step (c) or (d) in order to obtain the desired % of dry matter by total weight of malt sprouts fraction extract;
f. And optionally a sterilization step.

Advantageously, in the above-mentioned process, the hydrolysis is carried out at a temperature of 20° C. to 70° C., depending on the enzyme which is used.

In a particular embodiment, the process of preparation of malt sprouts fraction extract according to the invention and wherein the malt sprouts fraction extract comprises at least 0.5%, and less than 1.1% of dry matter by total weight of the malt sprouts fraction extract, comprises the following steps:
a. Dilution of isolated malt sprouts fractions enriched in anyone of rootlets, acrospires, hulk, dust and mixtures thereof in aqueous phase, preferably within a ratio from 1/4 to 1/15, preferably 1/5, 1/7, 1/10 or 1/12, to obtain diluted malt sprouts fractions;
b. Diffusion of these diluted malt sprouts fractions in aqueous phase followed by a finishing step at pH<6 or >8 and a thermal treatment at 100° C. preferably for 20 minutes, to obtain two phases, an aqueous one and a solid one;
c. Separation of the aqueous phase from the solid phase, preferably by centrifugation, to recover the aqueous phase;
d. Optionally microfiltration of the aqueous phase on plate filters to remove fine insoluble particles with a lower size than 1 μm, preferably lower than 0.8 μm and more preferably from 0.2 μm to 0.8 μm;
e. Optionally, treatment of the aqueous phase obtained in step (c) or (d) in order to obtain a % of dry matter from 0.5 to 1.1% by total weight of malt sprouts fraction extract;
f. And optionally a sterilization step.

In a particular embodiment, the process of preparation of malt sprouts fraction extract according to the invention and wherein the malt sprouts fraction extract comprises at least 1.1%, and less than 3.5% of dry matter by total weight of the malt sprouts fraction extract, comprises the following steps:
a. Dilution of isolated malt sprouts fractions enriched in anyone of rootlets, acrospires, hulk, dust and mixtures thereof in aqueous phase, preferably within a ratio from 1/4 to 1/15, preferably 1/5, 1/7, 1/10 or 1/12, to obtain diluted malt sprouts fractions;
b. Diffusion of these diluted malt sprouts fractions in aqueous phase followed by a finishing step at pH<6 or >8 and a thermal treatment at 100° C. preferably for 20 minutes, to obtain two phases, an aqueous one and a solid one;
c. Separation of the aqueous phase from the solid phase, preferably by centrifugation, to recover the aqueous phase;
d. Optionally microfiltration of the aqueous phase on plate filters to remove fine insoluble particles with a lower size than 1 μm, preferably lower than 0.8 μm and more preferably from 0.2 μm to 0.8 μm;
e. Optionally, treatment of the aqueous phase obtained in step (c) or (d) in order to obtain a % of dry matter from 1.1 to 3.5% by total weight of malt sprouts fraction extract;
f. And optionally a sterilization step.

In a particular embodiment, the process of preparation of malt sprouts fraction extract according to the invention and wherein the malt sprouts fraction extract comprises at least 3.5%, and less than 5% of dry matter by total weight of the malt sprouts fraction extract, comprises the following steps:
a. Dilution of isolated malt sprouts fractions enriched in anyone of rootlets, acrospires, hulk, dust and mixtures thereof in aqueous phase, preferably within a ratio from 1/4 to 1/15, preferably 1/5, 1/7, 1/10 or 1/12, obtain diluted malt sprouts fractions;
b. Diffusion of these diluted malt sprouts fractions in aqueous phase followed by a finishing step at pH<6 or >8 and a thermal treatment at 100° C. preferably for 20 minutes, to obtain two phases, an aqueous one and a solid one;
c. Separation of the aqueous phase from the solid phase, preferably by centrifugation, to recover the aqueous phase;
d. Optionally microfiltration of the aqueous phase on plate filters to remove fine insoluble particles with a lower size than 1 μm, preferably lower than 0.8 μm and more preferably from 0.2 μm to 0.8 μm;
e. Optionally, treatment of the aqueous phase obtained in step (c) or (d) in order to obtain a % of dry matter from 3.5 to 5% by total weight of malt sprouts fraction extract;
f. And optionally a sterilization step.

In a particular embodiment, the process of preparation of malt sprouts fraction extract according to the invention and wherein the malt sprouts fraction extract comprises at least 5%, and less than 7% of dry matter by total weight of the malt sprouts fraction extract, comprises the following steps:
a. Dilution of isolated malt sprouts fractions enriched in anyone of rootlets, acrospires, hulk, dust and mixtures thereof in aqueous phase, preferably within a ratio from 1/4 to 1/15, preferably 1/5, 1/7, 1/10 or 1/12, obtain diluted malt sprouts fractions;
b. Diffusion of these diluted malt sprouts fractions in aqueous phase followed by a finishing step at pH<6 or >8 and a thermal treatment at 100° C. preferably for 20 minutes, to obtain two phases, an aqueous one and a solid one;
c. Separation of the aqueous phase from the solid phase, preferably by centrifugation, to recover the aqueous phase;
d. Optionally microfiltration of the aqueous phase on plate filters to remove fine insoluble particles with a lower size than 1 μm, preferably lower than 0.8 μm and more preferably from 0.2 μm to 0.8 μm;
e. Optionally, treatment of the aqueous phase obtained in step (c) or (d) in order to obtain a % of dry matter from 5 to 7% by total weight of malt sprouts fraction extract;
f. And optionally a sterilization step.

In a particular embodiment, the process of preparation of malt sprouts fraction extract according to the invention and wherein the malt sprouts fraction extract comprises at least 7%, and less than 10% of dry matter by total weight of the malt sprouts fraction extract, comprises the following steps:
a. Dilution of isolated malt sprouts fractions enriched in anyone of rootlets, acrospires, hulk, dust and mixtures thereof in aqueous phase, preferably within a ratio from 1/4 to 1/15, preferably 1/5, 1/7, 1/10 or 1/12, to obtain diluted malt sprouts fractions;
b. Diffusion of these diluted malt sprouts fractions in aqueous phase followed by a finishing step at pH<6 or >8 and a thermal treatment at 100° C. preferably for 20 minutes, to obtain two phases, an aqueous one and a solid one;
c. Separation of the aqueous phase from the solid phase, preferably by centrifugation, to recover the aqueous phase;
d. Optionally microfiltration of the aqueous phase on plate filters to remove fine insoluble particles with a lower size than 1 μm, preferably lower than 0.8 μm and more preferably from 0.2 μm to 0.8 μm;
e. Optionally, treatment of the aqueous phase obtained in step (c) or (d) in order to obtain a % of dry matter from 7 to 10% by total weight of malt sprouts fraction extract;
f. And optionally a sterilization step.

In a particular embodiment, the process of preparation of malt sprouts fraction extract according to the invention and wherein the malt sprouts fraction extract comprises at least 10%, and less than 15% of dry matter by total weight of the malt sprouts fraction extract, comprises the following steps:
a. Dilution of isolated malt sprouts fractions enriched in anyone of rootlets, acrospires, hulk, dust and mixtures thereof in aqueous phase, preferably within a ratio from 1/4 to 1/15, preferably 1/5, 1/7, 1/10 or 1/12, to obtain diluted malt sprouts fractions;
b. Diffusion of these diluted malt sprouts fractions in aqueous phase followed by a finishing step at pH<6 or >8 and a thermal treatment at 100° C. preferably for 20 minutes, to obtain two phases, an aqueous one and a solid one;
c. Separation of the aqueous phase from the solid phase, preferably by centrifugation, to recover the aqueous phase;
d. Optionally microfiltration of the aqueous phase on plate filters to remove fine insoluble particles with a lower size than 1 μm, preferably lower than 0.8 μm and more preferably from 0.2 μm to 0.8 μm;
e. Optionally, treatment of the aqueous phase obtained in step (c) or (d) in order to obtain a % of dry matter from 10 to 15% by total weight of malt sprouts fraction extract;
f. And optionally a sterilization step.

In a particular embodiment, the process of preparation of malt sprouts fraction extract according to the invention and wherein the malt sprouts fraction extract comprises at least 15%, and less than 60% of dry matter by total weight of the malt sprouts fraction extract, comprises the following steps:
a. Dilution of isolated malt sprouts fractions enriched in anyone of rootlets, acrospires, hulk, dust and mixtures thereof in aqueous phase, preferably within a ratio from 1/4 to 1/15, preferably 1/5, 1/7, 1/10 or 1/12, to obtain diluted malt sprouts fractions;
b. Diffusion of these diluted malt sprouts fractions in aqueous phase followed by a finishing step at pH<6 or >8 and a thermal treatment at 100° C. preferably for 20 minutes, to obtain two phases, an aqueous one and a solid one;
c. Separation of the aqueous phase from the solid phase, preferably by centrifugation, to recover the aqueous phase;
d. Optionally microfiltration of the aqueous phase on plate filters to remove fine insoluble particles with a lower size than 1 μm, preferably lower than 0.8 μm and more preferably from 0.2 μm to 0.8 μm;
e. Treatment of the aqueous phase obtained in step (c) or (d) in order to obtain a % of dry matter from 15 to 60% by total weight of malt sprouts fraction extract;
f. And optionally a sterilization step.

In a particular embodiment, the process of preparation of malt sprouts fraction extract according to the invention and wherein the malt sprouts fraction extract comprises at least 60%, and less than 98% of dry matter by total weight of the malt sprouts fraction extract, comprises the following steps:
a. Dilution of isolated malt sprouts fractions enriched in anyone of rootlets, acrospires, hulk, dust and mixtures thereof in aqueous phase, preferably within a ratio from 1/4 to 1/15, preferably 1/5, 1/7, 1/10 or 1/12, to obtain diluted malt sprouts fractions;
b. Diffusion of these diluted malt sprouts fractions in aqueous phase followed by a finishing step at pH<6 or >8 and a thermal treatment at 100° C. preferably for 20 minutes, to obtain two phases, an aqueous one and a solid one;
c. Separation of the aqueous phase from the solid phase, preferably by centrifugation, to recover the aqueous phase;
d. Optionally microfiltration of the aqueous phase on plate filters to remove fine insoluble particles with a lower size than 1 μm, preferably lower than 0.8 μm and more preferably from 0.2 μm to 0.8 μm;
e. Treatment of the aqueous phase obtained in step (c) or (d) in order to obtain a % of dry matter from 60 to 98% by total weight of malt sprouts fraction extract;
f. And optionally a sterilization step.

In a particular embodiment, the process of preparation of malt sprouts fraction extract according to the invention and wherein the malt sprouts fraction extract comprises at least 7%, and less than 10% of dry matter by total weight of the malt sprouts fraction extract, comprises the following steps:
a. Dilution of isolated malt sprouts fractions enriched in anyone of rootlets, acrospires, hulk, dust and mixtures thereof in aqueous phase, preferably within a ratio from 1/4 to 1/15, preferably 1/5, 1/7, 1/10 or 1/12, to obtain diluted malt sprouts fractions;
b. Hydrolysis of these diluted malt sprouts fractions in the presence of exogenous enzymes, to obtain two phases, an aqueous one and a solid one;
c. Separation of the aqueous phase from the solid phase, preferably by centrifugation, to recover the aqueous phase;
d. Optionally microfiltration of the aqueous phase on plate filters to remove fine insoluble particles with a lower size than 1 μm, preferably lower than 0.8 μm and more preferably from 0.2 μm to 0.8 μm;
e. Optionally, treatment of the aqueous phase obtained in step (c) or (d) in order to obtain a % of dry matter from 7 to 10% by total weight of malt sprouts fraction extract;
f. And optionally a sterilization step.

In a particular embodiment, the process of preparation of malt sprouts fraction extract according to the invention and wherein the malt sprouts fraction extract comprises at least 10%, and less than 15% of dry matter by total weight of the malt sprouts fraction extract, comprises the following steps:
a. Dilution of isolated malt sprouts fractions enriched in anyone of rootlets, acrospires, hulk, dust and mixtures thereof in aqueous phase, preferably within a ratio from 1/4 to 1/15, preferably 1/5, 1/7, 1/10 or 1/12, to obtain diluted malt sprouts fractions;
b. Hydrolysis of these diluted malt sprouts fractions in the presence of exogenous enzymes, to obtain two phases, an aqueous one and a solid one;
c. Separation of the aqueous phase from the solid phase, preferably by centrifugation, to recover the aqueous phase;
d. Optionally microfiltration of the aqueous phase on plate filters to remove fine insoluble particles with a lower size than 1 μm, preferably lower than 0.8 μm and more preferably from 0.2 μm to 0.8 μm;
e. Optionally, treatment of the aqueous phase obtained in step (c) or (d) in order to obtain a % of dry matter from 10 to 15% by total weight of malt sprouts fraction extract;
f. And optionally a sterilization step.

In a particular embodiment, the process of preparation of malt sprouts fraction extract according to the invention and wherein the malt sprouts fraction extract comprises at least 15%, and less than 60% of dry matter by total weight of the malt sprouts fraction extract, comprises the following steps:
a. Dilution of isolated malt sprouts fractions enriched in anyone of rootlets, acrospires, hulk, dust and mixtures thereof in aqueous phase, preferably within a ratio from 1/4 to 1/15, preferably 1/5, 1/7, 1/10 or 1/12, to obtain diluted malt sprouts fractions;
b. Hydrolysis of these diluted malt sprouts fractions in the presence of exogenous enzymes, to obtain two phases, an aqueous one and a solid one;
c. Separation of the aqueous phase from the solid phase, preferably by centrifugation, to recover the aqueous phase;
d. Optionally microfiltration of the aqueous phase on plate filters to remove fine insoluble particles with a lower size than 1 μm, preferably lower than 0.8 μm and more preferably from 0.2 μm to 0.8 μm;
e. Treatment of the aqueous phase obtained in step (c) or (d) in order to obtain a % of dry matter from 15 to 60% by total weight of malt sprouts fraction extract;
f. And optionally a sterilization step.

In a particular embodiment, the process of preparation of malt sprouts fraction extract according to the invention and wherein the malt sprouts fraction extract comprises at least 60%, and less than 98% of dry matter by total weight of the malt sprouts fraction extract, comprises the following steps:
a. Dilution of isolated malt sprouts fractions enriched in anyone of rootlets, acrospires, hulk, dust and mixtures thereof in aqueous phase, preferably within a ratio from 1/4 to 1/15, preferably 1/5, 1/7, 1/10 or 1/12, to obtain diluted malt sprouts fractions;
b. Hydrolysis of these diluted malt sprouts fractions in the presence of exogenous enzymes, to obtain two phases, an aqueous one and a solid one;
c. Separation of the aqueous phase from the solid phase, preferably by centrifugation, to recover the aqueous phase;
d. Optionally microfiltration of the aqueous phase on plate filters to remove fine insoluble particles with a lower size than 1 μm, preferably lower than 0.8 μm and more preferably from 0.2 μm to 0.8 μm;
e. Treatment of the aqueous phase obtained in step (c) or (d) in order to obtain a % of dry matter from 60 to 98% by total weight of malt sprouts fraction extract;
f. And optionally a sterilization step.

In a particular embodiment, the process of preparation of malt sprouts fraction extract according to the invention and wherein the malt sprouts fraction extract comprises at least 0.5%, and less than 1.1% of dry matter by total weight of the malt sprouts fraction extract, comprises the following steps:
a. Dilution of isolated malt sprouts fractions enriched in anyone of rootlets, acrospires, hulk, dust and mixtures thereof in aqueous phase, preferably within a ratio from 1/4 to 1/15, preferably 1/5, 1/7, 1/10 or 1/12, to obtain diluted malt sprouts fractions;
b. Hydrolysis of these diluted malt sprouts fractions in the presence of exogenous enzymes, followed by a finishing step at pH<6 or >8 and thermal treatment at 100° C. preferably for 20 minutes, to obtain two phases, an aqueous one and a solid one;
c. Separation of the aqueous phase from the solid phase, preferably by centrifugation, to recover the aqueous phase;
d. Optionally microfiltration of the aqueous phase on plate filters to remove fine insoluble particles with a lower size than 1 μm, preferably lower than 0.8 μm and more preferably from 0.2 μm to 0.8 μm;
e. Optionally, treatment of the aqueous phase obtained in step (c) or (d) in order to obtain a % of dry matter from 0.5 to 1.1% by total weight of malt sprouts fraction extract;
f. And optionally a sterilization step.

In a particular embodiment, the process of preparation of malt sprouts fraction extract according to the invention and wherein the malt sprouts fraction extract comprises at least 1.1%, and less than 3.5% of dry matter by total weight of the malt sprouts fraction extract, comprises the following steps:
a. Dilution of isolated malt sprouts fractions enriched in anyone of rootlets, acrospires, hulk, dust and mixtures thereof in aqueous phase, preferably within a ratio from 1/4 to 1/15, preferably 1/5, 1/7, 1/10 or 1/12, to obtain diluted malt sprouts fractions;
b. Hydrolysis of these diluted malt sprouts fractions in the presence of exogenous enzymes, followed by a finishing step at pH<6 or >8 and thermal treatment at 100° C. preferably for 20 minutes, to obtain two phases, an aqueous one and a solid one;
c. Separation of the aqueous phase from the solid phase, preferably by centrifugation, to recover the aqueous phase;
d. Optionally microfiltration of the aqueous phase on plate filters to remove fine insoluble particles with a lower size than 1 μm, preferably lower than 0.8 μm and more preferably from 0.2 μm to 0.8 μm;
e. Optionally, treatment of the aqueous phase obtained in step (c) or (d) in order to obtain a % of dry matter from 1.1 to 3.5% by total weight of malt sprouts fraction extract;
f. And optionally a sterilization step.

In a particular embodiment, the process of preparation of malt sprouts fraction extract according to the invention and wherein the malt sprouts fraction extract comprises at least 3.5%, and less than 5% of dry matter by total weight of the malt sprouts fraction extract, comprises the following steps:
a. Dilution of isolated malt sprouts fractions enriched in anyone of rootlets, acrospires, hulk, dust and mixtures thereof in aqueous phase, preferably within a ratio from 1/4 to 1/15, preferably 1/5, 1/7, 1/10 or 1/12, to obtain diluted malt sprouts fractions;
b. Hydrolysis of these diluted malt sprouts fractions in the presence of exogenous enzymes, followed by a finishing step at pH<6 or >8 and thermal treatment at 100° C. preferably for 20 minutes, to obtain two phases, an aqueous one and a solid one;
c. Separation of the aqueous phase from the solid phase, preferably by centrifugation, to recover the aqueous phase;
d. Optionally microfiltration of the aqueous phase on plate filters to remove fine insoluble particles with a lower size than 1 μm, preferably lower than 0.8 μm and more preferably from 0.2 μm to 0.8 μm;
e. Optionally, treatment of the aqueous phase obtained in step (c) or (d) in order to obtain a % of dry matter from 3.5 to 5% by total weight of malt sprouts fraction extract;
f. And optionally a sterilization step.

In a particular embodiment, the process of preparation of malt sprouts fraction extract according to the invention and wherein the malt sprouts fraction extract comprises at least 5%, and less than 7% of dry matter by total weight of the malt sprouts fraction extract, comprises the following steps:
a. Dilution of isolated malt sprouts fractions enriched in anyone of rootlets, acrospires, hulk, dust and mixtures thereof in aqueous phase, preferably within a ratio from 1/4 to 1/15, preferably 1/5, 1/7, 1/10 or 1/12, to obtain diluted malt sprouts fractions;
b. Hydrolysis of these diluted malt sprouts fractions in the presence of exogenous enzymes, followed by a finishing step at pH<6 or >8 and thermal treatment at 100° C. preferably for 20 minutes, to obtain two phases, an aqueous one and a solid one;
c. Separation of the aqueous phase from the solid phase, preferably by centrifugation, to recover the aqueous phase;
d. Optionally microfiltration of the aqueous phase on plate filters to remove fine insoluble particles with a lower size than 1 μm, preferably lower than 0.8 μm and more preferably from 0.2 μm to 0.8 μm;
e. Optionally, treatment of the aqueous phase obtained in step (c) or (d) in order to obtain a % of dry matter from 5 to 7% by total weight of malt sprouts fraction extract;
f. And optionally a sterilization step.

In a particular embodiment, the process of preparation of malt sprouts fraction extract according to the invention and wherein the malt sprouts fraction extract comprises at least 7%, and less than 10% of dry matter by total weight of the malt sprouts fraction extract, comprises the following steps:
a. Dilution of isolated malt sprouts fractions enriched in anyone of rootlets, acrospires, hulk, dust and mixtures thereof in aqueous phase, preferably within a ratio from 1/4 to 1/15, preferably 1/5, 1/7, 1/10 or 1/12, to obtain diluted malt sprouts fractions;
b. Hydrolysis of these diluted malt sprouts fractions in the presence of exogenous enzymes, followed by a finishing step at pH<6 or >8 and thermal treatment at 100° C. preferably for 20 minutes, to obtain two phases, an aqueous one and a solid one;
c. Separation of the aqueous phase from the solid phase, preferably by centrifugation, to recover the aqueous phase;
d. Optionally microfiltration of the aqueous phase on plate filters to remove fine insoluble particles with a lower size than 1 μm, preferably lower than 0.8 μm and more preferably from 0.2 μm to 0.8 μm;
e. Optionally, treatment of the aqueous phase obtained in step (c) or (d) in order to obtain a % of dry matter from 7 to 10% by total weight of malt sprouts fraction extract;
f. And optionally a sterilization step.

In a particular embodiment, the process of preparation of malt sprouts fraction extract according to the invention and wherein the malt sprouts fraction extract comprises at least 10%, and less than 15% of dry matter by total weight of the malt sprouts fraction extract, comprises the following steps:
a. Dilution of isolated malt sprouts fractions enriched in anyone of rootlets, acrospires, hulk, dust and mixtures thereof in aqueous phase, preferably within a ratio from 1/4 to 1/15, preferably 1/5, 1/7, 1/10 or 1/12, to obtain diluted malt sprouts fractions;
b. Hydrolysis of these diluted malt sprouts fractions in the presence of exogenous enzymes, followed by a finishing step at pH<6 or >8 and thermal treatment at 100° C. preferably for 20 minutes, to obtain two phases, an aqueous one and a solid one;
c. Separation of the aqueous phase from the solid phase, preferably by centrifugation, to recover the aqueous phase;
d. Optionally microfiltration of the aqueous phase on plate filters to remove fine insoluble particles with a lower size than 1 μm, preferably lower than 0.8 μm and more preferably from 0.2 μm to 0.8 μm;
e. Optionally, treatment of the aqueous phase obtained in step (c) or (d) in order to obtain a % of dry matter from 10 to 15% by total weight of malt sprouts fraction extract;
f. And optionally a sterilization step.

In a particular embodiment, the process of preparation of malt sprouts fraction extract according to the invention and wherein the malt sprouts fraction extract comprises at least 15%, and less than 60% of dry matter by total weight of the malt sprouts fraction extract, comprises the following steps:
a. Dilution of isolated malt sprouts fractions enriched in anyone of rootlets, acrospires, hulk, dust and mixtures thereof in aqueous phase, preferably within a ratio from 1/4 to 1/15, preferably 1/5, 1/7, 1/10 or 1/12, to obtain diluted malt sprouts fractions;
b. Hydrolysis of these diluted malt sprouts fractions in the presence of exogenous enzymes, followed by a finishing step at pH<6 or >8 and thermal treatment at 100° C. preferably for 20 minutes, to obtain two phases, an aqueous one and a solid one;
c. Separation of the aqueous phase from the solid phase, preferably by centrifugation, to recover the aqueous phase;
d. Optionally microfiltration of the aqueous phase on plate filters to remove fine insoluble particles with a lower size than 1 μm, preferably lower than 0.8 μm and more preferably from 0.2 μm to 0.8 μm;
e. Treatment of the aqueous phase obtained in step (c) or (d) in order to obtain a % of dry matter from 15 to 60% by total weight of malt sprouts fraction extract;
f. And optionally a sterilization step.

In a particular embodiment, the process of preparation of malt sprouts fraction extract according to the invention and wherein the malt sprouts fraction extract comprises at least 60%, and less than 98% of dry matter by total weight of the malt sprouts fraction extract, comprises the following steps:
a. Dilution of isolated malt sprouts fractions enriched in anyone of rootlets, acrospires, hulk, dust and mixtures thereof in aqueous phase, preferably within a ratio from 1/4 to 1/15, preferably 1/5, 1/7, 1/10 or 1/12, to obtain diluted malt sprouts fractions;
b. Hydrolysis of these diluted malt sprouts fractions in the presence of exogenous enzymes, followed by a finishing step at pH<6 or >8 and thermal treatment at 100° C. preferably for 20 minutes, to obtain two phases, an aqueous one and a solid one;
c. Separation of the aqueous phase from the solid phase, preferably by centrifugation, to recover the aqueous phase;
d. Optionally microfiltration of the aqueous phase on plate filters to remove fine insoluble particles with a lower size than 1 μm, preferably lower than 0.8 μm and more preferably from 0.2 μm to 0.8 μm;
e. Treatment of the aqueous phase obtained in step (c) or (d) in order to obtain a % of dry matter from 60 to 98% by total weight of malt sprouts fraction extract;
f. And optionally a sterilization step.

Another object of the invention is a composition consisting of a malt sprouts fraction extract according to the invention wherein the malt sprouts fraction extract is such as obtained by a method of preparation comprising the following steps:
a. Dilution of isolated malt sprouts fractions enriched in anyone of rootlets, acrospires, hulk, dust and mixtures thereof in aqueous phase, preferably within a ratio from 1/4 to 1/15, preferably 1/5, 1/7, 1/10 or 1/12, to obtain diluted malt sprouts fractions;
b. Diffusion of these diluted malt sprouts fractions in aqueous phase, followed by a finishing step at pH<6 or >8 and a thermal treatment at 100° C. preferably for 20 minutes, to obtain two phases, an aqueous one and a solid one;
c. Separation of the aqueous phase from the solid phase, preferably by centrifugation, to recover the aqueous phase;
d. Optionally microfiltration of the aqueous phase on plate filters to remove fine insoluble particles with a lower size than 1 μm, preferably lower than 0.8 μm and more preferably from 0.2 μm to 0.8 μm;
e. Optionally, treatment of the aqueous phase obtained in step (c) or (d) in order to obtain the desired % of dry matter by total weight of malt sprouts fraction extract;
f. And optionally a sterilization step.

Advantageously, in the above-mentioned process, the diffusion is carried out at a temperature of 20° C. to 50° C.

Another object of the invention is a composition consisting of a malt sprouts fraction extract according to the invention wherein the malt sprouts fraction extract comprises at least 7%, and less than 98% of dry matter by total weight of the malt sprouts fraction extract, and is such as obtained by a method of preparation comprising the following steps:
a. Dilution of isolated malt sprouts fractions enriched in anyone of rootlets, acrospires, hulk, dust and mixtures thereof in aqueous phase, preferably within a ratio from 1/4 to 1/15, preferably 1/5, 1/7, 1/10 or 1/12, to obtain diluted malt sprouts fractions;
b. Hydrolysis of these diluted malt sprouts fractions in the presence of exogenous enzymes, to obtain two phases, an aqueous one and a solid one;
c. Separation of the aqueous phase from the solid phase, preferably by centrifugation, to recover the aqueous phase;
d. Optionally microfiltration of the aqueous phase on plate filters to remove fine insoluble particles with a lower size than 1 μm, preferably lower than 0.8 μm and more preferably from 0.2 μm to 0.8 μm;
e. Optionally, treatment of the aqueous phase obtained in step (c) or (d) in order to obtain the desired % of dry matter by total weight of malt sprouts fraction extract;
f. And optionally a sterilization step.

Advantageously, in the above-mentioned process, the hydrolysis is carried out at a temperature of 20° C. to 70° C., depending on the enzyme which is used.

Another object of the invention is a composition consisting of a malt sprouts fraction extract according to the invention wherein the malt sprouts fraction extract is such as obtained by a method of preparation comprising the following steps:
a. Dilution of isolated malt sprouts fractions enriched in anyone of rootlets, acrospires, hulk, dust and mixtures thereof in aqueous phase, preferably within a ratio from 1/4 to 1/15, preferably 1/5, 1/7, 1/10 or 1/12, to obtain diluted malt sprouts fractions;
b. Hydrolysis of these diluted malt sprouts fractions in the presence of exogenous enzymes, followed by a finishing step at pH<6 or >8 and thermal treatment at 100° C. preferably for 20 minutes, to obtain two phases, an aqueous one and a solid one;
c. Separation of the aqueous phase from the solid phase, preferably by centrifugation, to recover the aqueous phase;
d. Optionally microfiltration of the aqueous phase on plate filters to remove fine insoluble particles with a lower size than 1 μm, preferably lower than 0.8 μm and more preferably from 0.2 μm to 0.8 μm;
e. Optionally, treatment of the aqueous phase obtained in step (c) or (d) in order to obtain the desired % of dry matter by total weight of malt sprouts fraction extract;
f. And optionally a sterilization step.

Advantageously, in the above-mentioned process, the hydrolysis is carried out at a temperature of 20° C. to 70° C., depending on the enzyme which is used.

Another object of the invention is a composition comprising at least a malt sprouts fraction extract according to the invention or a malt sprouts fraction extract obtained by a process according to the invention, and at least one ingredient selected from additional ingredients.

According to the invention, “additional ingredients” are selected from compounds stimulating the cellular growth, compounds stimulating the fermentation, compounds contributing to flavor enhancement in food or enabling to reduce salt in food, and preferably include yeast extracts, vegetal peptones, malt extracts, and mixtures thereof.

According to the invention, additional ingredients are selected from protein hydrolysates, organic acids, mineral acids, mineral salts, sugars, polyols, and mixtures thereof.

In a particular embodiment, in a composition according to the invention, the additional ingredients are selected from protein hydrolysates, organic acids, mineral acids, mineral salts, sugars, polyols and mixtures thereof.

According to the invention, the term “protein hydrolysates” refers to a mixture of peptides and amino acids prepared by hydrolysing a primary protein source with acid, alkali, or enzyme. These protein hydrolysates are generally presented in the form of powders or liquid preparations, fully soluble in water, developed for some industrial uses, and generally ready for use for various laboratory applications (e.g. media for microbiological controls). They provide a nitrogen source for bacteriological, industrial and specialized media for microbial, plant, animal and insect cell cultures on both laboratory and industrial scale. However, in many instances protein hydrolysates also provide vitamins, residual carbohydrates, minerals, and different anions and cations.

In a particular embodiment, in a composition according to the invention, the additional ingredients are protein hydrolysates.

In a particular embodiment, the composition according to the invention contains a protein mixture wherein the protein hydrolysates originate from soy, rapeseed, pea, alfalfa, wheat protein, casein, gelatin, meat, yeast.

In a particular embodiment, the composition according to the invention comprising at least a malt sprouts fraction extract according to the invention and at least one additional ingredient selected from protein hydrolysates, is characterized at least by:
Total Nitrogen content from 45 to 140 g/kg of dry matter
Free Amino Nitrogen content from 10 to 60 g/kg of dry matter, and
Nucleic Acid Derivatives such as purines and pyrimidines bases in an amount from 2 to 10 g/kg of dry matter.
PUPY content from 0.5 to 8 g/kg of dry matter
Ratio PUPY/Nucleic Acid Derivatives higher than 20%.

The above-mentioned composition according to the invention is characterized by a ratio PUPY/Nucleic Acid Derivatives from 20 to 100%, in particular from 20 to 30%, from 20 to 40%, from 20 to 50%, from 20 to 60%, or from 20 to 70%.

In a particular embodiment, a composition according to the invention can comprise a malt sprouts fraction extract and additional nitrogen compounds containing DAN, respectively obtained by a process according to the invention applied separately to malt sprout fractions and primary protein sources rich in nucleic acids.

Another object of the invention is a composition such as obtained by the process of preparation of a protein mixture containing malt sprouts fraction extract and additional nitrogen compounds, according to the invention and comprising the following steps:
a. Diffusion of diluted malt sprouts fractions and primary protein source, in aqueous phase, optionally followed by a finishing step at pH<6 or >8 and a thermal treatment at 100° C. preferably for 20 minutes, to obtain two phases, an aqueous one and a solid one;
   or hydrolysis of diluted malt sprouts fractions and primary protein source, in the presence of exogenous enzymes, optionally followed by a finishing step at pH<6 or >8 and thermal treatment at 100° C. preferably for 20 minutes, to obtain two phases, an aqueous one and a solid one;
b. Separation of the aqueous phase from the solid phase, preferably by centrifugation, to recover the aqueous phase;
c. Optionally microfiltration of the aqueous phase on plate filters to remove fine insoluble particles with a lower size than 1 μm, preferably lower than 0.8 μm and more preferably from 0.2 μm to 0.8 μm;
d. Optionally, treatment of the aqueous phase obtained in step (c) or (d) in order to obtain the desired % of dry matter by total weight of malt sprouts fraction extract;
e. And optionally a sterilization step, characterized at least by:
   Total Nitrogen content from 50 to 130 g/kg of dry matter
   Free Amino Nitrogen content from 15 to 50 g/kg of dry matter, and
   Nucleic Acid Derivatives such as purines and pyrimidines bases in an amount from 3 to 9 g/kg of dry matter.
   PUPY content from 1 to 8 g/kg of dry matter
   Ratio PUPY/Nucleic Acid Derivatives higher than 20%.

Advantageously, in the above-mentioned process, the diffusion is carried out at a temperature of 20° C. to 50° C.

Advantageously, in the above-mentioned process, the hydrolysis is carried out at a temperature of 20° C. to 70° C., depending on the enzyme which is used.

The above-mentioned composition according to the invention is characterized by a ratio PUPY/Nucleic Acid Derivatives from 20 to 100%, in particular from 20 to 30%, from 20 to 40%, from 20 to 50%, from 20 to 60%, or from 20 to 70%.

In the case of primary protein sources having a high content of nucleic acids, a composition, such as described above, obtained by the process of preparation of a protein mixture containing malt sprouts fraction extract and additional nitrogen compounds, according to the invention, can have substantially the same characterization (in total N, FAN, DAN, PUPY) as a composition according to the invention comprising a malt sprouts fraction extract and additional nitrogen compounds containing DAN respectively obtained by a process according to the invention applied separately to malt sprout fractions and primary protein sources rich in nucleic acids.

In a particular embodiment, the composition according to the invention is a food ingredient, particularly a flavor enhancer, used in food for animals or humans.

According to the invention, the term “flavor enhancer” refers to a substance added to food for animals or humans in order to enhance or intensify the flavor of food and/or the palatability of the food and/or enabling to reduce salt in food.

In a particular embodiment, the composition according to the invention is a culture and/or fermentation medium.

The cell culture requires a specific medium, named culture medium, which is generally an aqueous formulation including carbon and nitrogen sources, mineral salts, oligo elements and optionally different additional nutrients and growth factors.

A fermentation medium is adapted to the culture of a specific micro-organism and/or to the production of a specific metabolite.

It is advantageous to provide purines and pyrimidines bases in a cell culture medium in particular for the production of micro-organisms, microbial metabolites or fermentation, whereas cell culture medium are usually only enriched in Free Amino Nitrogen.

It is also an additional advantage to provide ribose phosphate and deoxyribose phosphate or ribose and deoxyribose.

In a particular embodiment, the composition according to the invention is a food ingredient, particularly a flavor enhancer, used in food for animals or humans, or a culture and/or fermentation medium.

Another object of the invention is the use of a composition comprising or consisting of a malt sprouts fraction extract according to the invention or a malt sprouts fraction extract obtained by a process according to the invention, as nitrogen source and/or sugar source and/or protein source.

The inventors have developed new malt sprouts fraction extracts obtained from fractions of germ plant cells in a growth phase (rootlets and acrospires), dried in the drying of malt, and characterized by high nutritional value ingredients, especially nitrogen compounds including free amino nitrogen compounds and purines and pyrimidines bases. These compounds are of particular interest for heterotrophic growth during germination, and have applications in various cell cultures. The inventors have demonstrated that the use of these new malt sprout fraction extracts containing purine (adenine (or hypoxanthine resulting from the deamination of adenine), guanine) and pyrimidines (cytosine, uracil, thymine) in a culture medium is a favorable factor in the development and the multiplication of cells, which is not considered in the prior art relating to malt sprouts fraction extracts. The use of malt sprouts fraction extracts of the invention as sources of purine and pyrimidine bases in the fermentation media also provides the microorganisms the possibility to use them directly and bypass the classical biosynthesis process, which can be energetically interesting.

Several malt sprouts fraction extracts have been developed and their interest and efficiency for several applications, in particular for cell culture, has been demonstrated.

Another object of the invention is the use of a composition comprising or consisting of a malt sprouts fraction extract according to the invention or a malt sprouts fraction extract obtained by a process according to the invention, in a fermentation process, in particular in a fermentation process using mycetes including actinomycetes, fungi, yeasts and/or bacteriae.

According to the invention, fermentation is limited to the culture of micro-organisms, both aerobically and anaerobically, to produce those micro-organisms (microbial biomass) or some products (metabolites) resulting from their metabolism. Metabolites include an extremely broad range of substances from very simple molecules as ethanol, lactic acid, amino acids to very complex ones as biopolymers, functional proteins, enzymes, antibiotics.

Another object of the invention is the use of a composition comprising or consisting of a malt sprouts fraction extract according to the invention or a malt sprouts fraction extract obtained by a process according to the invention, in production of micro-organisms, such as yeasts, bacteriae, mycetes including actinomycetes, fungi, useful in particular in sanitary, food and/or agronomy or pharmaceuticals applications.

Another object of the invention is the use of a composition comprising or consisting of a malt sprouts fraction extract according to the invention or a malt sprouts fraction extract obtained by a process according to the invention, in production of metabolites by fermentation.

Another object of the invention is the use of a composition comprising or consisting of a malt sprouts fraction extract according to the invention or a malt sprouts fraction extract obtained by a process according to the invention, in culture of microbial cells, plant cells and/or animal and/or human cells.

Such cell cultures processes allow to produce a wide range of different metabolites as antibiotics when using microbial cells, and as antibodies when using animal and/or human cells.

In the sense of the invention, the terms ‘cell culture’ or ‘culture of cells’ refer globally to biological or microbiological techniques to grow and multiply living unicellular or multicellular organisms, (yeast, bacteria, microbial, animal, human or plant cells or tissues) either in the laboratory or industrially (fermentation, production of biomass and/or metabolites or other components).

Another object of the invention is the use of a composition comprising or consisting of a malt sprouts fraction extract according to the invention or a malt sprouts fraction extract obtained by a process according to the invention, in germination of plant or grains.

According to the invention, the term “germination” refers to the process of seeds developing into new plants. First the seed grows a root to access water underground. Next, the shoots, or growth above ground, begin to appear. The seed sends a shoot towards the surface, where it will grow leaves to harvest energy from the sun.

Another object of the invention is the use of a composition comprising or consisting of a malt sprouts fraction extract according to the invention or a malt sprouts fraction extract obtained by a process according to the invention, in foods for animals or humans, in particular for pets or fishes as animals.

According to the invention, the terms “food for animals” or “animal foods” refer to pet food and feed.

Another object of the invention is the use of a composition comprising or consisting of a malt sprouts fraction extract according to the invention or a malt sprouts fraction extract obtained by a process according to the invention, as a flavor enhancer.

In a particular embodiment, malt sprouts fraction extract according to the invention or a malt sprouts fraction extract obtained by a process according to the invention, or a composition according to the invention, are used to improve the fermentation of sourdough, and thereby generating a specific flavor in the final bread.

In a particular embodiment, a composition comprising or consisting of malt sprouts fraction extract according to the invention or a malt sprouts fraction extract obtained by a process according to the invention, are used as nitrogen source and/or sugar source and/or protein source, or in a fermentation process, in particular in a fermentation process using mycetes including actinomycetes, fungi, yeasts and/or bacteriae, or in production of micro-organisms, such as yeasts, bacteriae, mycetes including actinomycetes, fungi, useful in particular in sanitary, food and/or agronomy or pharmaceuticals applications, or in production of metabolites by fermentation.

In a particular embodiment, a composition comprising or consisting of malt sprouts fraction extract according to the invention or a malt sprouts fraction extract obtained by a process according to the invention, are used in culture of microbial cells, plant cells and/or animal and/or human cells, or in germination of plant or grains, or in foods for animals or humans, in particular for pets or fishes as animals, or as a flavor enhancer in food for animals or humans.

Another object of the invention is a culture and/or fermentation process using a living micro-organism comprising at least a step wherein a composition comprising or consisting of a malt sprouts fraction extract according to the invention or a malt sprouts fraction extract obtained by a process according to the invention, is added to the culture and/or fermentation medium or replaces it as a substitute.

Another object of the invention is a kit of products for culture and/or fermentation process comprising:
(i) A composition comprising or consisting of a malt sprouts fraction extract according to the invention or a malt sprouts fraction extract obtained by a process according to the invention,
(ii) A culture and/or fermentation medium or alternatively all ingredients to prepare extemporaneously the said culture and/or fermentation medium,
(iii) Optionally additional ingredients,
(iv) And optionally a notice of use.

Another object of the invention is a kit of products for foods for animals or humans comprising:
(i) A composition comprising or consisting of a malt sprouts fraction extract according to the invention or a malt sprouts fraction extract obtained by a process according to the invention,
(ii) A food product or all ingredients to prepare extemporaneously the food product,
(iii) Optionally additional ingredients,
(iv) And optionally a notice of use.

In a particular embodiment, the kit of products for culture and/or fermentation process comprises:
(i) A composition comprising or consisting of a malt sprouts fraction extract according to the invention or a malt sprouts fraction extract obtained by a process according to the invention,
(ii) A culture and/or fermentation medium or alternatively all ingredients to prepare extemporaneously the said culture and/or fermentation medium,
(iii) Optionally additional ingredients,
(iv) And optionally a notice of use,
or the kit of products for foods for animals or humans, comprises:
(i) A composition comprising or consisting of a malt sprouts fraction extract according to the invention or a malt sprouts fraction extract obtained by a process according to the invention,
(ii) A food product or all ingredients to prepare extemporaneously the food product,
(iii) Optionally additional ingredients,
(iv) And optionally a notice of use.

The examples hereafter demonstrate that malt sprouts fraction extracts can improve the culture of micro-organisms to produce yeasts (examples 3, 4.2.) and lactic acid bacteria (examples 4.3., 6, 8, 9). They can also improve the production of lactic acid which is a fermentation metabolite (e.g. production of lactic acid in example 4.3.1. and 4.3.2.). The applications of malt sprouts fraction extracts should not be understood to be limited to such examples.

The use of malt sprouts fraction extracts can be generalized to the formulation of media adapted:
to the different cultures of micro-organisms: mycetes (yeasts and fungi), bacteria, actinomycetes . . . for applications as ferments, koji, probiotics, bio protection, bio control . . . .
to the fermentation of microbial metabolites: alcohols, organic acids, amino acids, enzymes, functional proteins, exopolysaccharides, antibiotics, . . . .

Malt sprouts fraction extracts can also be used in the production of fermented foods, as for instance production of sourdough from cereal flours to obtain sourdough breads.

In addition, malt sprouts fraction extracts can be considered as new ingredients in media formulations for the culture of eukaryotes cells.

Media composition is of the upmost importance for animal cells culture as complex components from animal origin are required for the growth and multiplication of those cells.

Because of their vegetal origin as growing plant cells, malt sprouts fraction extracts are well adapted to the in vitro culture of vegetal cells or tissues. Malt sprouts fraction extracts are sources of minerals, including micro and oligo elements, free or phosphorylated C5 sugars, FAN, DAN, PUPY and different phytohormons. malt sprouts fraction extracts are interesting new ingredients to formulate media for the culture of vegetal cells or tissues.

Other embodiments of the invention are hereafter disclosed. For the understanding of these embodiments, the following definitions of some expressions are indicated, which are in no way in contradiction with the previously mentioned definitions.

The state of the art highlights some of the many attempts at the research stage and has been for over thirty years to create malt sprouts extracts and study their applications, especially for growing microorganisms. However there are no preparations of malt sprouts extracts on the market for commercial applications for cell cultures as well as any other applications. This absence in the market of malt sprouts extracts preparations is intrinsically linked to the characteristics and properties of the extracts described in the prior art, which limits their applications and therefore the interests of obtaining them. In particular:
1) The great variability of raw materials used for the production of extracts; which directly impacts the quality and consistency of extracts, while users want fixed and constant quality products in time;
2) The excessive dilution of the aqueous extracts (soluble solids concentration lower or equal to 3.5% w/w);
3) The low degree of extraction of the soluble material and nitrogenous matter (crude protein N×6.25);
4) Insufficient stability of some diluted extracts makes them unsuitable as such to the storage, transport or handling practices in economic conditions;
5) Finally, the variability of the composition of the extracts makes them less attractive as ingredients used as yeast extract or soy peptones whose compositions are better controlled.

The term “rootlet” used generically by the profession actually refers to a mixture of different co-products generated during the malting, containing various proportions: malt dust, and sometimes barley dust, envelope fragments (husk or hull), fragments of broken grains, roots (rootlets) and acrospires or fragments thereof. The real roots and the acrospires from the germ (malt sprouts) are the first visible components of seedlings. The coarse mixture of these co-products is marketed as such or as granules (pellets) in animal feed under the “malt sprouts” which is somewhat excessive given the presence proportionally more or less other components.

As an indicative composition of malt sprouts, in particular barley malt sprouts, used as initial raw material for the extraction process according to the invention, it may advantageously contain:
80-96% of Dry matter (d.m)
20-35% d.m. of Crude protein (N×6.25)
43-57% d.m. total fibers from which 40-50% insoluble fibers (cellulose, hemicellulose, lignine) and 2-5% of soluble fibers;
7-17% d.m. total carbohydrates from which 4-7% starch and 7-10% sugars
<1% d.m fats
5-7% d.m ash.

A first aspect of the invention concerns a malt sprouts extract characterized at least by:
(i) Total Nitrogen content higher than 45 g/kg of dry matter
(ii) Free Amino Nitrogen content higher than 10 g/kg of dry matter, and
(iii) Nucleic acid derivatives such as purines and pyrimidines bases in an amount higher than 2 g/kg of dry matter.

In a particular embodiment, the amount of total nitrogen will range from 50 g/kg to 90 g/kg of dry matter, preferably from 50 g/kg to 75 g/kg of dry matter (diffusion process) and from 60 g/kg to 85 g/kg of dry matter (hydrolysis process).

In a particular embodiment, the amount of Free Amino Nitrogen (FAN) will range from 12 g/kg to 40 g/kg of dry matter, preferably from 12 g/kg to 20 g/kg of dry matter (diffusion process) and from 20 g/kg to 40 g/kg of dry matter (hydrolysis process).

In a particular embodiment, the amount of Nucleic Acid Derivatives (DAN) will range from 3 to 25 g/kg of dry matter, preferably from 3 to 24 g/kg of dry matter (diffusion process) and from 8 g/kg to 24 g/kg of dry matter (hydrolysis process).

In a particular embodiment, the malt sprout extract according to the invention will have a percentage of dry matter ranging from 1.1% to 98% of dry matter by total weight of the malt sprout extract.

In particular, the malt sprout extract according to the invention will have a percentage of dry matter ranging from 1.1% to 97% of dry matter (optimized diffusion process) by total weight of the malt sprout extract, and from 7.5% to 97% of dry matter (simple diffusion process) by total weight of the malt sprout extract.

In particular, the malt sprout extract according to the invention will have a percentage of dry matter ranging from 1.1% to 97% of dry matter (hydrolysis process) by total weight of the malt sprout extract, and from 3% to 97% of dry matter (optimized hydrolysis process) by total weight of the malt sprout extract.

Another object of the invention is a method of preparation of a malt sprouts extract as defined above, comprising the following steps:
(a) Isolation of malt sprouts fractions enriched in anyone of rootlets, acrospires, hulk, dust and mixtures thereof,
(b) Dilution of theses fractions in aqueous phase, preferably in water,
(c) Diffusion in aqueous phase, or alternatively hydrolysis; preferably followed by a finishing step as disclosed herein;
(d) Separation of the aqueous phase from the solid phase,
(e) Optionally a concentration step,
(f) And optionally a sterilization step.

Another aspect of the invention is a composition comprising at least a malt sprout extract according to the invention and a carrier comprising at least one ingredient selected from additional actives and/or additives.

In another aspect, the invention concerns the uses of said malt sprout extract or the composition containing it.

The new malt sprouts extracts of the invention are then an alternative to currently commercially available ingredients for cell culture. They can firstly reduce formulation costs of media compositions, but on the other hand, they contain nutrients, growth factors and other useful complex compounds for growth and cell proliferation. The features and capabilities make them particularly interesting to stimulate the activity of microorganisms and thus increase yield and/or productivity of industrial fermentations. They can also be used in the formulation of various culture media for laboratory. Moreover, these new products can also have an interest in various applications of cell cultures. These new products present also an interest as flavor enhancer and/or nutrient in foods for animals or humans, in particular for pets, fishes or insects or bugs as animals.

In a particular embodiment, the industrial interest concentrated extracts of malt fractions in liquid (slurry) or powder forms also lies in the valuation that can make malting expanding possibilities for use of its byproducts to segments of higher added value than animal feed. They can provide additional economic contribution to the malt house.

The man skilled in the art will be able to use either the diluted, concentrated and/or powder form, and in the efficient amount, depending on the applications and searched results.

A first object of the invention concerns a malt sprouts extract characterized at least by:
(i) Total Nitrogen content higher than 45 g/kg of dry matter
(ii) Free Amino Nitrogen content higher than 10 g/kg of dry matter, and
(iii) Nucleic acid derivatives such as purines and pyrimidines bases in an amount higher than 2 g/kg of dry matter.

In a preferred embodiment, the malt sprouts extract comprises at least 1.1%, in particular at least 3.5% and less than 7.5% of dry matter by total weight of malt sprouts extract and containing at least:
(i) Total nitrogen content higher than 45 g/kg of dry matter;
(ii) Free amino nitrogen content higher or equal than 12 g/kg of dry matter; and
(iii) Nucleic acid derivatives such as purine and pyrimidine bases content higher or equal than 3 g/kg of dry matter.

For example, a malt sprout extract comprising 3.5% of dry matter by total weight of malt sprouts extracts, also named a diluted malt sprout extract or juice according to the invention, will contain approximately 1.6% of total nitrogen, 0.42% of free amino nitrogen and 0.1% of nucleic acid derivatives by total weight of the malt sprout extract.

Such malt sprouts extract comprising at least 1.1%, in particular at least 3.5% and less than 7.5% of dry matter by total weight of malt sprout extracts is named according to the invention as ‘diluted malt sprouts extract’, as they contain a low amount of dry matter by total weight of extract.

In a particular embodiment, the ‘diluted malt sprout extract’ is not obtained by a method of preparation named ‘simple diffusion method’ comprising the following steps:
(a) Isolation of malt sprouts fractions enriched in anyone of rootlets, acrospires, hulk, dust and mixtures thereof, at the end of the malt drying step, and preferably grinding these fractions;
(b) Dilution of theses fractions in aqueous phase, preferably in water within a ratio equal or higher than 1/4, in particular between 1/8 and 1/12 (malt sprouts fractions/water),
(c) Diffusion in aqueous phase (simple diffusion without any finishing step);
(d) Separation of the aqueous phase from the solid phase, preferably by centrifugation,
(e) Optionally microfiltration of the aqueous phase on plate filters to remove fine insoluble particles with a lower size than 1 μm, preferably lower than 0.7 μm and more preferably from 0.2 μm to 0.7 μm;
(f) Treatment of the aqueous phase obtained in step (d) or (e) in order to obtain a % of dry matter higher than 1.1%, preferably higher than 3.5% by weight of total weight, preferably not higher than 7.5% of dry matter by total weight of malt sprout extract,
(g) And optionally a sterilization step.

In a preferred embodiment, the ‘diluted malt sprouts extract’ is obtained by a method of preparation comprising the following steps:
(a) Isolation of malt sprouts fractions enriched in anyone of rootlets, acrospires, hulk, dust and mixtures thereof, at the end of germination or at the malt drying step, and preferably grinding these fractions;
(b) Dilution of theses fractions in aqueous phase, preferably within a ratio equal or higher than 1/4, in particular between 1/8 and 1/12,
(c) Diffusion in aqueous phase followed by a finishing step at pH<6 or >8 and a thermal treatment at 100° C. preferably for 20 minutes, or alternatively hydrolysis in the presence of exogenous enzymes preferably followed by a finishing step at pH<6 or >8 and thermal treatment at 100° C. preferably for 20 minutes;
(d) Separation of the aqueous phase from the solid phase, preferably by centrifugation,
(e) Optionally microfiltration of the aqueous phase on plate filters to remove fine insoluble particles with a lower size than 1 μm, preferably lower than 0.8 μm and more preferably from 0.2 μm to 0.7 μm;
(f) Treatment of the aqueous phase obtained in step (d) or (e) in order to obtain a % of dry matter higher than 1.1%, preferably higher than 3.5% of dry matter by total weight, preferably not higher than 7.5% by weight of total weight of malt sprout extract,
(g) And optionally a sterilization step.

These preferred methods of preparation of the diluted malt sprout extracts encompass the named ‘optimized diffusion method’ (with a finishing step), the ‘simple hydrolysis method’ and the ‘optimized hydrolysis method’ (with a finishing step).

The finishing step with specific pH conditions (<6 or >8) and T° C. conditions (thermal treatment), as disclosed further in the description, is advantageous as it increases the solubility of the ingredients of interest.

In another preferred embodiment, the malt sprouts extract comprises at least 7.5%, preferably at least 10% of dry matter by total weight of malt sprouts extract and containing at least:
(i) Total nitrogen content higher than 45 g/kg of dry matter;
(ii) Free amino nitrogen content higher or equal than 12 g/kg of dry matter; and
(iii) Nucleic acid derivatives such as purine and pyrimidine bases content higher or equal than 3 g/kg of dry matter.

For example, a malt sprout extract comprising 15% of dry matter by total weight of malt sprouts extracts, also named a concentrated malt sprout extract or concentrate liquid or slurry (syrup) according to the invention, will contain approximately 6.75% of total nitrogen, 1.8% of free amino nitrogen and 0.45% of nucleic acid derivatives by total weight of the malt sprout extract.

Such malt sprouts extract comprising at least 7.5%, preferably at least 10% of dry matter by total weight of the malt sprout extract, is named according to the invention as ‘concentrated malt sprouts extract as juice’.

In a preferred embodiment, concentrated malt sprouts extract is in a solid form, preferably a powder, comprising more than 85% of dry matter by total weight of malt sprouts extract and containing at least:
(i) Total nitrogen content higher than 45 g/kg of dry matter;
(ii) Free amino nitrogen content higher or equal than 12 g/kg of dry matter; and
(iii) Nucleic acid derivatives such as purine and pyrimidine bases content higher or equal than 3 g/kg of dry matter.

In another preferred embodiment, concentrated malt sprouts extract is in a liquid or slurry form, preferably a liquid or slurry concentrate, comprising 10 to 85% of dry matter by weight of total weight and containing at least:
(i) Total nitrogen content higher than 45 g/kg of dry matter;
(ii) Free amino acid content higher or equal than 12 g/kg of dry matter; and
(iii) Nucleic acid derivatives such as purine and pyrimidine bases content higher or equal than 3 g/kg of dry matter.

For example, a malt sprout extract comprising 95% of dry matter by total weight of malt sprouts extracts, also named a powder malt sprouts extract according to the invention, will contain approximately 42.75% of total nitrogen, 11.4% of free amino nitrogen and 2.85% of nucleic acid derivatives by total weight of the malt sprout extract.

The malt sprouts extract according to the invention may advantageously comprise additionally at least one compound selected from:
Antioxydants, in particular glutathion,
Enzymes, in particular selected from amylases, phosphodiesterases and mixtures thereof,
Carbohydrates
Proteins
Soluble Fibers
Plant hormones,
B vitamins and minerals,
   and mixtures thereof.

As examples of antioxydants, it may include glutathion, antioxydant phenolic compounds, and mixtures thereof.

As examples of enzymes, it may include in particular amylases, proteases, phosphodiesterases and mixtures thereof,

As examples of carbohydrates, it preferably includes starch, sugars and mixtures thereof.

As examples of proteins, it preferably includes hordeins, globulins, albumins and mixtures thereof.

As examples of soluble fibers, it preferably includes cellulose, hemicellulose, lignin, beta-glucanes, arabinoxylanes and mixtures thereof, preferably beta-glucanes, arabinoxylanes and mixtures thereof.

As examples of plant hormones, it preferably includes auxins, cytokinins, gibberellins and mixtures thereof.

As examples B vitamins, it preferably includes B3 Niacin, B5 panthotenic acid, B6 pyridoxine, B1 thiamin, B2 riboflavin, and mixtures thereof.

As examples of minerals, it preferably includes potassium, phosphore, sulfure, magnesium, chlorides, iron, and mixtures thereof.

These highly functional ingredients are of interest, in particular:
High nitrogen content, amino acid profile, high FAN content, high diastatic power, purines and pyrimidines, antioxydants, B vitamins and minerals as growth factors, for cell culture and fermentation applications;
High diastatic power and high 5′-phosphodiesterase content for flavor enhancer production;
High fiber content and green/grass taste coming from acrospires, for feed.

In a particular embodiment, malt sprouts extract of the invention is obtained from malted cereal selected from barley, wheat, rye, spelt, corn, millet sorghum, oat, triticale, rice and mixtures thereof, preferably barley.

In a preferred embodiment, the malt sprouts extract is a barley malt sprouts extract.

As examples of barley varieties, mention may be made to the varieties selected from Sebastian, Azurel, Etincel, Irina and mixtures thereof.

Advantageously, the malt sprout extract of the invention is obtained from an aqueous extraction of malt sprouts fractions of barley comprising a content higher than 50%, preferably higher than 60%, more preferably higher than 65% and even more comprised between 65 and 85% of rootlets and acrospires by total weight of malt sprout fractions.

These sub-fractions as described above may be obtained by the following protocol:

Material: Vibratory sieve shaker Retsch AS200
Weight around 100 g of malt sprouts fractions (note the exact weight)
Select the following ‘sieves’: bottom-250-560-1000 μm and assemble them (top to bottom) from the biggest to the smallest screen
Put the raw material on the first sieve and close the Retsch
Activate according to following program:
   1 minute at amplitude 50
   3 minutes at amplitude 60
   1 minute at amplitude 63
Weight the 4 sub-fractions recovered.

In a preferred embodiment, the malt sprouts extract of the invention is obtained from an aqueous extraction of malt sprouts fractions from one or more cereals, in particular of barley, whose fractions contain more than 20% of sub-fractions having a granulometry ranging from 315-500 μm, preferably more than 50% of sub-fractions having a granulometry ranging from 315-500 μm.

The sub-fractions as described above may be obtained by the following protocol:

Material: Vibratory sieve shaker Fritsch
Weight around 100 g of malt sprouts fractions (note the exact weight)
Select the following ‘sieves’: bottom-200-315-500-800-1000-2000 μm and assemble them (top to bottom) from the biggest to the smallest screen
Put the raw material on the first sieve and close the Fritsch
Activate 15 minutes.
Weight the 7 sub-fractions recovered.

Another object of the invention is a method of preparation of a malt sprouts extract as defined above, comprising the following steps:
isolating at the end of germination or at the malt drying step a fraction enriched in parts of the grain mainly from the germ (rootlets “true”, acrospires and fragments thereof);
extracting in aqueous phase that fraction of malt, including preferably a finishing step as disclosed herein,
separating the aqueous phase from the solid phase and to remove fine residual particles from the aqueous phase,
optionally concentrating the dilute aqueous phase formulated as such or formulated with ingredients or additives to obtain a stable liquid formula,
optionally drying the concentrated aqueous phase or without the addition of ingredients or additives, to obtain a solid preparation.

In a preferred embodiment, a method of preparation of a malt sprouts extract as defined above, comprises the following steps:
(a) Isolation of malt sprouts fractions enriched in anyone of rootlets, acrospires, hulk, dust and mixtures thereof, at the end of germination or at the malt drying step, and preferably grinding these fractions;
(b) Dilution of theses fractions in aqueous phase, preferably within a ratio equal or higher than 1/10, such as 1/8, more preferably within a ratio equal to 1/10;
(c) Diffusion in aqueous phase, or alternatively hydrolysis and in the presence of exogenous enzymes;
(d) Preferably a finishing step at pH<6 or pH >8 and thermal treatment at 100° C., preferably for 20 minutes
(e) Separation of the aqueous phase from the solid phase by centrifugation, or pressing.
(f) Optionally microfiltration of the aqueous phase on plate filters to remove fine insoluble particles with a lower size than 1 μm, preferably lower than 0.7 μm and more preferably from 0.2 μm to 0.7 μm;
(g) Treatment of the aqueous phase obtained in step (e) or (f) in order to obtain a % of dry matter higher than 3.5% of dry matter by total weight, preferably higher than 7.5% of dry matter by total weight of malt sprout extract,
(h) And optionally a sterilization step.

Isolation and Selection of Fractions

The malt sprouts fractions enriched in anyone of rootlets, acrospires, hulk, dust and mixtures thereof, are obtained at the end of germination or at the malt drying step according well known methods, in particular derived from kilning or deculming and/or obtained from techniques such as bolting, screening. In a particular embodiment, the malt sprouts fractions may content higher than 50%, preferably higher than 60%, more preferably higher than 65% and even more comprised between 65 and 85% of rootlets and acrospires by total weight of fractions.

These malt sprouts fractions may optionally be sieved as disclosed above to obtain selected malt sprouts fractions with more than 20% of sub-fractions having a granulometry ranging from 315-500 μm, preferably more than 50% of sub-fractions having a granulometry ranging from 315-500 μm.

These selected fractions are then grinded, for example using a Bauknecht grinder (level 0).

Dilution

The fractions are diluted in aqueous phase, preferably in water, in a specific ratio ranging from 1/12 to 1/4 (1 malt sprout fraction 1/12 to 4 water) with usual stirred vessels. Lower dilutions require adequate mixing devices.

In a particular embodiment for a malt sprout extract obtained by a ‘diffusion method’, the ratio malt sprout fractions/water will range from 1/12 to 1/4, preferably from 1/5 to 1/10.

In a particular embodiment for a malt sprout extract obtained by a ‘hydrolysis method’, the ratio malt sprout fractions/water will range from 1/12 to 1/8, preferably from 1/11 to 1/9 and more preferably a ratio equal to 1/10.

The aqueous phase comprises preferably water as organic solvent.

Diffusion or Hydrolysis

According to one embodiment, the fractions are diluted in aqueous phase for obtaining a diffusion.

According to another preferred embodiment, the fractions are diluted in aqueous phase then submitted to a hydrolysis in the presence of exogenous enzymes.

The pH of the hydrolysis reaction is adjusted to the optimal pH of selected enzymes. The optimal pH of xylanase/cellulose are close to 4-5, and the optimal pH of protease are close to 5-6. So 2 pH have been used: stable at initial pH 5 then pH 7 after 2 h of hydrolysis.

The concentrations of each enzyme were been calculated to be in excess, and in particular:
Xylanases: 1.5%/Dry Matter
Cellulases: 2%/Cellulose
Proteases 0.3%/Dry Matter

The enzymes are preferably added in 2 steps, at t0, the xylanases and cellulases mixture then at 2 h, the proteases.

The temperature of the hydrolysis reaction is ranging from 50° C. to 60° C. Preferably, the hydrolysis is made for 4 h in total including 2 h at 60° C. then the temperature is decreased at 50° C. during 2 h.

Inactivation

The inactivation of exogenous enzymes occurs when a high temperature is applied at the end of the hydrolysis reaction, preferably a temperature of 80° C. for at least 5 minutes.

Finishing Step

The finishing step is made at pH<6 or >8 and a thermal treatment at 100° C. preferably for 20 minutes

This step is advantageous as it increases the solubility of the ingredients of interest.

Separation and Rinsing

The solid/liquid separation is made by centrifugation, preferably on Servall RC12BP at 4000 g, 10 minutes at 20° C.

After recovery of the juice, the cake is rinsed with osmotic water at room temperature with a volume of the cake, then a second centrifugation is done (4000 g for 10 minutes).

Filtration

The extract is then filtered on plate filters having specific pore size, preferably 0.7 μm and 0.2 μm. Advantageously, the filters are cellulose filters KDS15 and S80-0.7 μm and 0.2 μm respectively.

Concentration

In a preferred embodiment, the extract is concentrated, for example on Heidolph rotovapor (20 L), at 60° C. until reach 13% of dry matter.

Drying

Finally and in order to obtain an extract in a powder form, the extract is dried, for example on Büchi 190 spray-dryer, at maximum aspiration, with inlet temperatures 117-120° C. and outlet temperatures 78-82° C.

In a preferred embodiment, the treatment of the aqueous phase in step g) comprises a step of concentration to obtain a liquid or slurry concentrate with a % of dry matter higher than 10% by weight of total weight.

The concentration of an extract is generally made by evaporation, but reverse osmosis is an option to pre-concentrate to a dry matter content up to 15%.

In a particular embodiment, the method of preparation of a malt sprouts extract in liquid form, in particular in liquid or slurry concentrate, comprises a diffusion in step c) realized for at least 1 hour at a temperature ranging from 20° C. to 50° C., preferably 2 hours at 20° C.

In another preferred embodiment, the treatment of the aqueous phase in step g) comprises a step of drying of the aqueous phase, or alternatively a step of concentration of the aqueous phase followed by a step of drying of the said liquid concentrate, to obtain a solid preparation, preferably a powder with a % of dry matter higher than 85% by weight of total weight.

In a particular embodiment, the method of preparation of a malt sprouts extract in solid form, in particular in powder form, comprises an hydrolysis in step c) realized for 6 to 8 hours, preferably 4 hours at a temperature ranging from 60° C. to 50° C., pH from 5 to 7, with one or more enzymes selected from cellulases, xylanases, proteases, nucleases, phosphohydrolases and mixtures thereof, followed by an enzyme thermal inactivation step and further by a stirring step at 50° C.

Another object of the present invention is a composition comprising at least a malt sprouts extract as defined above or a malt sprouts extract obtained by method of preparation as defined above, and a carrier comprising at least one ingredient selected from additional actives and/or additives.

The term composition according to the invention also includes media composition.

In a particular embodiment, the additional actives are selected from compounds stimulating the cellular growth, compounds stimulating the fermentation, formulation aids or excipients and the additional additives are selected from organic acids, mineral acids, mineral salts, sugars, polyols and mixtures thereof.

As additional actives, it preferably includes yeast extracts, vegetal peptones, malt extracts, and mixtures thereof.

As examples of organic acids, it preferably includes acetic, lactic, propionic, sorbic, malic, benzoic, hydroxybenzoic acid, and their salts, and mixtures thereof.

As mineral acids, it preferably includes phosphoric, nitric, nitrous SO2, sulfites bisulfites, metabisulfites, and mixtures thereof.

As mineral salts, it preferably includes NaCl.

As sugars, it preferably includes glucose, maltose, sucrose and mixtures thereof.

As polyols, it preferably includes glycerol, sorbitol, mannitol, and mixtures thereof.

The compositions can include combinations of these additives and should be adapted to applications.

Advantageously, the composition may also comprise formulation aids or excipients such as emulsifiers, thickeners, gelling agents, stabilizers, coloring agents, and preservatives.

In a preferred embodiment, the composition of the invention is a composition for food, in particular a food product for animals or humans.

The food product may be in any form adapted to an oral administration such as liquid, slurry or solid form.

The food product may generally comprise usual ingredients for such application, in particular chosen from sugars, fats, vitamins, mineral salts, excipients.

The malt sprout extract is of interest in animal and/or human food product as flavor enhancer and for its nutrient properties as nitrogen source and/or sugar source and/or protein source.

The malt sprout extract may be present in a food product in a content ranging from 0.1 to 0.8% of dry matter by weight of food product, preferably in a content ranging from 0.2 to 0.4% of dry matter by weight of food product.

In another preferred embodiment, the composition of the invention is a medium, and in particular a culture and/or fermentation medium.

In particular, the composition of the invention is a growth medium for Petri dishes.

The medium may be in any form adapted to a cell culture or fermentation process; it may be in a liquid, gelled or solid form, or alternatively in a powder form that may be diluted in water extemporaneously before use.

A culture medium is a medium containing all ingredients necessary for the culture and growth of cells and/or tissues, in particular animal or plant cells or tissues.

As example, a medium for cell culture may comprise a carbon source such as glucose, water, various salts, preferably hormones and growth factors, and a source of amino acids and nitrogen.

A fermentation medium is a medium containing all ingredients necessary for the fermentation process using micro-organisms, in particular a fermentation process using mycetes including actinomycetes, fungi, yeasts and/or bacteriae.

As example, a medium for fermentation may comprise a carbon source such as glucose, water, various salts, and a source of amino acids and nitrogen.

The man skilled in the art will adapt the amount of malt sprout extract (dry matter) depending the application and the searched result.

The malt sprout extract may be present in a cell culture medium in a content ranging from 1% to 10% of dry matter by weight of the cell culture medium, preferably in a content ranging from 5% to 8% of dry matter by weight of the cell culture medium.

The malt sprout extract may be present in a fermentation medium in a content ranging from 1% to 10% of dry matter by weight of the fermentation medium, preferably in a content ranging from 5% to 8% of dry matter by weight of the fermentation medium.

Another object of the present invention is the use of a malt sprouts extract as defined above, or a malt sprout extract obtained by a method of preparation as defined above, or a composition as defined above, as nitrogen source and/or sugar source and/or protein source.

The said malt sprout extract may be used as a partial or total substituent of nitrogen source and/or sugar source and/or protein source of the composition (either medium or food product).

In a preferred embodiment, the malt sprouts extract as defined above, or a malt sprout extract obtained by a method of preparation as defined above, or a composition as defined above is used, in a fermentation process, in particular in a fermentation process using mycetes including actinomycetes, fungi, yeasts and/or bacteriae.

The malt sprout extract according to the invention may be used for fermentation process in solid or liquid media.

In particular, the malt sprouts extracts of the invention can be used in media of industrial fermentation process, from the initial phases of preparation of inocula and successive propagation (during which it is essential to ensure better health, the best vitality and activity microorganisms used), until the phase in the production fermenter. These fermentations concern in particular fermented food by yeasts such as the production of beer or liquor distilled or not; fermented food by lactic acid bacteria such as the production of yoghourt and dairy products; or fermented food by combinations of these micro-organisms such as the production of bread products.

In another preferred embodiment, the malt sprouts extract as defined above, or a malt sprout extract obtained by a method of preparation as defined above, or a composition as defined above is used in production of micro-organisms, such as mycetes including actinomycetes, fungi, yeasts and/or bacteriae, useful in particular in sanitary, food, agronomy applications and/or pharmaceutical applications.

As examples of micro-organisms production; it preferably includes the production of:
yeast for bakery, brewery, winery and other fermentations of nutritional yeast, probiotic yeast or for sanitary applications;
lactic acid bacteria used as starter cultures or enzymes for the food, probiotics, or for sanitary applications;
mushrooms food ferments, spores or mycelium for the production of edible mushroom;
bacteria, yeast or fungi used for bio protection, bio-control or for agronomic purposes.
Specific bacteria for pharmaceutical applications.

In another preferred embodiment, the malt sprouts extract as defined above, or a malt sprout extract obtained by a method of preparation as defined above, or a composition as defined above is used in production of metabolites by fermentation.

As metabolites, it preferably includes:
primary metabolites and in particular organic acids and amino acids;
secondary metabolites and in particular antibiotics or other various molecules: vitamins, nucleotides, enzymes, bio-polymers, and bio-insecticides.

In another preferred embodiment, the malt sprouts extract as defined above, or a malt sprout extract obtained by a method of preparation as defined above, or a composition as defined above is used in culture of microbial cells, plant cells and/or animal cells.

The presence in the malt sprout extracts according to the invention of vitamins, nutrients and growth factors, has very interesting functional consequences in cell cultures, and in particular an improved cellular physiological state: the cell with the environmental factors essential to its development improves its anabolic balance sheet and thereby less energy for its development or multiplication.

This results in a better cell activity in interaction with its culture medium which is measured by:
the increased growth rate (μ, h−1) or number generation per unit time that quantify cell proliferation rate;
the increase in yield expressed as the ratio of cell biomass produced (X, g/L) or of the expected product of metabolism (P, g/L) to the quantity of substrate consumed (usually the main carbon substrate (S, g/L). it may also perform cell counts at the end of cultivation (N, number/mL) to quantify the multiplication factor of the cell population and to reduce the amount of consumed substrate;
the increase in productivity as measured by the amount of cells or of product obtained per unit volume (or reactor used) and per unit time; and/or
the increase in cell viability as measured by the mortality rate in the stationary phase during which there is an essential nutrient limitation (in general the main carbon substrate).

The malt sprouts extracts according to the invention can be used as ingredients in formulations or ready-made liquid culture media, agar, or solid for cell cultures for purposes of research or monitoring, particularly for microbiological counts flores generic (total flora, mold yeast, lactic bacteria . . . ) or specific.

Applications also relate to cell cultures or from plant or animal tissues.

In another preferred embodiment, the malt sprouts extract as defined above, or a malt sprout extract obtained by a method of preparation as defined above, or a composition as defined above is used in the process of germination of plant grains or seedlings.

In particular, they can be used to activate or control the development of germination or the development of seedlings. They have agricultural or industrial applications, including malting or for the production of sprouts for nutritional purposes.

In another preferred embodiment, the malt sprouts extract as defined above, or a malt sprout extract obtained by a method of preparation as defined above, or a composition as defined above is used as a flavor enhancer, in particular flavor enhancer in food.

In particular, malt sprout extracts of the invention can replace yeast extracts used in many foods, and may be combined with glutamate.

In another preferred embodiment, the malt sprouts extract as defined above, or a malt sprout extract obtained by a method of preparation as defined above, or a composition as defined above is used in foods for animals or humans, in particular for pets or fishes as animals.

In particular, the malt extract fractions of the invention may be incorporated in feed formulations for pets as palatant factor due to their high nitrogen compounds including derivatives of nucleotides and molecules coming from Maillard reactions.

The malt extract fractions of the invention can be incorporated into food for farmed fish because of their high nitrogen compounds including derivatives of nucleic acids, preferably IMP and GMP (Inosine monophosphate and Guanosine monophosphate). In particular, they provide essential nitrogen nutrient factors whose levels are generally limiting in the existing protein sources, including vegetable protein, soybean, rapeseed, for example.

The malt sprout extract of the invention may also be used in aquaculture or entomoculture, and in particular as substitute of fish flour in aquaculture.

Another object of the present invention concerns a culture and/or fermentation process using a living micro-organism comprising at least a step wherein a malt sprouts extract as defined above, or a malt sprout extract obtained by a method of preparation as defined above, or a composition as defined above, is added to the culture and/or fermentation medium or replaces it as a substitute.

The present invention also concerns a kit of products for culture and/or fermentation process comprising:

A malt sprouts extract as defined above, or a malt sprout extract obtained by a method of preparation as defined above, or a composition as defined above,
(ii) A culture and/or fermentation medium or alternatively all ingredients to prepare extemporaneously the said culture and/or fermentation medium,
(iii) Optionally additional active and/or additives ingredients,
(iv) And optionally a notice of use.

Another object of the present invention is a kit of products for foods for animals or humans comprising:
(i) A malt sprouts extract as defined above, or a malt sprout extract obtained by a method of preparation as defined above, or a composition as defined above,
(ii) A food product or all ingredients to prepare extemporaneously the food product,
(iii) Optionally additional active and/or additives ingredients,
(iv) And optionally a notice of use.

In a particular embodiment, a diluted malt sprouts extract as juice according to the invention may not be used in a fermentation process when the said diluted malt sprouts extract is obtained by a simple diffusion (without a finishing step).

A diluted malt sprouts extract according to the invention, when obtained by a simple diffusion (without a finishing step) may be used in one or several following applications:
in production of bacteriae, mycetes including actinomycetes, fungi, useful in particular in sanitary, food and/or agronomy or pharmaceutical applications,
in production of metabolites by fermentation.
in culture of microbial cells, plant cells and/or animal cells,
in germination of plant or grains,
as a flavor enhancer.
in foods for animals or humans, in particular for pets or fishes as animals,
   and may be included in the composition or kit prepared to implement these applications.

The malt sprouts extracts according to the invention in form of juice, liquid concentrate or powder, may be further diluted before application, and depending the applications in which they will be used.

The following examples illustrate the invention without limiting the scope thereof. Unless otherwise stated, the percentages are expressed by weight.

### FIGURES

FIG. 1: Schematic illustration of malt sprouts, with (1) representing an acrospire (=germ), (2) representing rootlets, (3) representing the husk (=hull), (4) representing a malted barley grain. A malt sprout is constituted of a mix of (1)+(2)+(3)+malt dust.

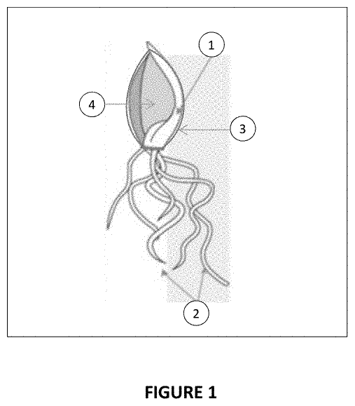

### EXAMPLES

### Example 1: Preparation of Malt Sprout Fraction Extracts in Form of Powder (from Simple Diffusion (Powder 1A) and Simple Hydrolysis Processes (Powder 1B))

The malt sprout fraction used to produce malt sprout fraction extract in this example is characterized in Tables 1 and 2, is ground using a grinder Bauknecht (level 0). The ground fraction is divided into two part of 12 kg to perform extractions in the presence of water in the ratio 1/10 (1 malt fraction/9 water).

TABLE 1
Physical characterization of malt sprout fraction MSF 1
MSF1 - Physical characterization

| | |
|---|---|
| From crop | 2014 |

Fractions composition (in %)

| | |
|---|---|
| Husk + whole grains | 10 |
| Rootlets + Acrospires | 72 |
| Dust | 18 |

TABLE 2
Physico-chemical analysis of MSF1

| | Units | Values |
|---|---|---|
| Dry u matter | % | 97.6 |
| Total nitrogen (N) | g/100 g | 5.1 |
| Total protein (N*6.25) | g/100 g | 31.7 |

The extractions are carried out in a 200 L reactor equipped with a double envelope. One of the slurry is maintained for 2 h at 20° C. at the natural pH of the mixture. The other is maintained for 2 h 30 min at 50° C. and pH 7.5 in the presence of enzymes, then the temperature is raised to 72° C. and maintained at this value until the end of the extraction, a term total of 4 hours. Following a preliminary study, the choice of enzymes fell on a combination of two proteases preparations Sumizyme LP and Protease Plus.

In both cases, the final mixture is heated at 80° C. for 5 min. Each of the mixtures thus obtained is then subjected to the same protocol of liquid-solid separation comprising:
A first filtration with a filter press Choquenet (P8329 canvas TC) in the presence of diatomaceous earth (DIC/S) under 4 bar pressure, followed by rinsing the filter cake with a volume of osmotic water in a ratio 1/4 of the volume of the first juice and final compaction of the cake to 7.5 bar.
Centrifugation (RC12BP+at 4000 g, 10 min, 20° C.) after the additional filter press was carried out in the second case due to a too large content of residual solid particles.
The juices were then subjected to a second filtration in two steps using a plate filter (KDS15 filters and S80 at 0.7 and 0.2 microns, respectively).
The juices (also named diluted malt sprouts fraction extracts) such obtained are characterized in the following Table 3:

TABLE 3
Characterization of the diluted malt sprout fraction extracts
obtained after plate-filter step.

| | MSFE1-A | MSFE1-B |
|---|---|---|
| Dry matter dm (%) | 1.14 | 3.98 |
| Total nitrogen (g/kg dm) | 58.77 | 65.07 |

After plate-filter step the two malt sprout fraction extracts have been concentrated until 8.4% dm and 11.25% dm respectively, before the step of drying on Büchi 190 spray-dryer, at maximum aspiration, feed:200 ml/h (level 4), inlet T° C. 117-120° C. (level 4.5-5), outlet T° C. 78-82° C.
2 powders of malt sprout fraction extracts have been recovered following the spray-drying step, characterized in Table 4.

TABLE 4
Characterization of powdered malt sprout fraction extracts.

| | Powder 1A | Powder 1B |
|---|---|---|
| Dry matter (%) | 96.7 | 97.5 |
| Total nitrogen (g/kg dm) | 55.1 | 63.8 |
| Total FAN (g/kg dm) | 14.8 | 28.7 |
| Total DAN (g/kg dm) | 4.0 | 4.6 |
| Total PUPY (g/kg dm) | 3.8 | 4.4 |
| Ratio PUPY/DAN | 97.7 | 95.5 |

### Example 2: Preparation of Malt Sprout Fraction Extract (from Simple Hydrolysis Process) in the Form of Syrup

The malt sprout fraction (MSF2) used to produce malt sprout fraction extract in this example is characterized in Tables 5 and 6, is ground using a grinder Bauknecht (level 0). This 14 kg of ground fraction is used to perform extraction in the presence of water in the ratio 1/10.

TABLE 5
Physical characterization of MSF2
MSF2-Physical characterization

| | |
|---|---|
| From crop | 2015 |

Fractions composition (in %)

| | |
|---|---|
| Husk + whole grains | 11 |
| Rootlets + Acrospires | 74 |
| Dust | 15 |

TABLE 6
Physico-chemical analysis of MSF2

| Malt fractions blend | Units | Values |
|---|---|---|
| Moisture | % | 6.3 |
| Total nitrogen (N) | g/100 g | 4.9 |
| Total protein (N*6.25) | g/100 g | 30.3 |

The extraction is carried out in a 200 L reactor equipped with a double envelope. The aqueous extract is adjusted at pH 5 with H₂SO₄ (95%), cellulases and xylanases are added, temperature is maintained for 2 h at 60° C. Then, pH of the aqueous extract is adjusted at 7 and proteases are added, temperature is decreased to 50° C. and maintained at this value until the end of the extraction, a term total of 4 hours.

At the end of extraction, the final mixture is heated at 80° C. for 5 min. The mixture thus obtained is then subjected to the following protocol of liquid-solid separation comprising:
A first filtration with a filter press Choquenet (P8329 canvas TC) in the presence of diatomaceous earth (DIC/S) under 4 bar pressure, followed by rinsing the filter cake with a volume of osmotic water in a ratio 1/4 of the volume of the first juice and final compaction of the cake to 7.5 bar.
Centrifugation (BTPX centrifuge+at 8000 g, sludge discharge frequency: 30 min, flow 100 liters/hour, 20° C.) after the additional filter press was carried out in the second case due to a too large content of residual solid particles.
The juice were then subjected to a second filtration in two steps using a plate filter (KDS15 filters and S80 at 0.7 and 0.2 microns, respectively).
The malt sprout fraction extract (MSFE2) such obtained is characterized in the following Table 7:

TABLE 7
Characterization of the MSFE2 obtained after plate-filter step.

| | MSFE2 |
|---|---|
| Dry matter (%) | 2.74 |
| Total nitrogen (g/kg dm) | 66.05 |

After plate-filter step the juice has been concentrated until 60% dm on Heidolph rotovapor (20 liters capacity) at 60° C.
This syrup (slurry concentrate) has been recovered following the concentration step and characterized in Table 8.

TABLE 8
Characterization of malt sprout fraction extract (MSFE2)
in syrup form.

| | MSFE2 (syrup form) |
|---|---|
| Dry matter dm (%) | 60.5 |
| Total nitrogen (g/kg dm) | 66.06 |
| Total FAN (g/kg dm) | 19.05 |
| Total DAN (g/kg dm) | 8.02 |
| Total PUPY (g/kg dm) | 7.05 |
| Ratio PUPY/DAN (%) | 87.9 |

### Example 3: Effect of Malt Sprout Fraction Extract in Powder (Produced from Diffusion Process) in Cell Culture Medium for Yeast

The malt sprout fraction extract in powder form (powder 1A, whose preparation is detailed in Example 1, and which is characterized in table 4), was used for the culture of Saccharomyces boulardii in liquid medium compared to control medium containing yeast extract (YE 0251PW-L Biospringer).

Each of the media used contained 1 g N/kg, 10 g of glucose/kg of mineral elements (KH2PO4, MgSO4 7H2O and a solution of trace elements respectively 10, 5.12 and 10 g/kg). The initial pH was adjusted to 5.5; after inoculation with the strain of S. boulardii (SuperSmart Saccharomyces boulardii—250 mg) they were incubated at 30° C. in Erlenmeyer stirring at 110 rpm.

The kinetics were followed to estimate the growth rate in the exponential phase (μ). Biomass X (yeast dry matter) produced after 24 h and 42 h of fermentation was measured after filtration (0.45μ) of samples from media, washing the filter cake and then drying at 105° C. for 24 hours.

The results are presented in the following Table 9:

TABLE 9
Productions cultures of S boulardii from yeast extract and
Malt sprout fraction extract (powder 1A).

| Media | μ (h⁻¹) | X 24 (g/kg) | X 42 (g/kg) |
|---|---|---|---|
| Control medium Yeast | 0.16 | 8.34 | 8.09 |
| Extract (YE) | | | |
| Powder 1A (Example 1) | 0.18 | 8.25 | 10.94 |

With YE control medium after 24 hours of culture, the biomass concentration is 8.34 g/L then decreased slightly 8.09 g/kg at 42 h culture. This finding is not comparable to results obtained with the powder 1 for which the biomass concentration increases between 24 and 42 h: 10.94 g/kg. At nitrogen iso-concentration (1 g/L to N) and on a sterilized medium after filtration under 0.2μ, using the same culture conditions, the malt sprout fraction extract (Powder 1A) allows an almost identical growth in a medium composed of yeast extract but lead to a higher biomass production when fermentation continues beyond 24 hours.

### Example 4: Effect of Malt Sprout Fraction Extracts in Powder Form (Produced from Simple Hydrolysis Process) in Cell Culture Media

### 4.1: Preparation of Malt Sprout Fraction Extract in Powder Form (from Simple Hydrolysis Process)

TABLE 10
Physico-chemical analysis of malt
sprout fraction used for the example 4
MSF3

| | | |
|---|---|---|
| Dry matter | % | 93.7 |
| Total protein (N* 6.25) | g/100 g | 29.6 |

A malt sprout fraction extract in powder form (identified powder 2204) was obtained from the malt sprout fraction MSF3 according to the following protocol:
The malt sprout fraction MSF3 is suspended to 1/10 in water in the presence of cellulolytic enzymes (SD 800) and hemicellulolytic enzymes (GC 220). The initial pH of the mixture is adjusted to 5, its temperature at 60° C. The suspension was then maintained at 60° C. for 105 min and then the temperature is brought to 50° C. over 30 min. Meanwhile, after 2 hours of extraction, the pH is adjusted to 7 and treated with proteases (Plus PIB and Sumizyme LP) in the suspension. The extraction continues 105 minutes at 50° C., then the suspension is heated to 80° C. 5 minutes and cooled down to 50° C.
The suspension is kept 2 hours at that temperature.
The suspension thus obtained is then subjected to the following protocol of liquid-solid separation comprising:
A first liquid-solid separation step, the centrifugation (RC12BP+at 4000 g, 10 min, 20° C.) in order to remove the large solid particles.
The juice obtained from centrifuge was then subjected to a second filtration in two steps using a plate filter (KDS15 filters and S80 at 0.7 and 0.2 microns, respectively).
After plate-filter step the juice has been concentrated until 13% dm, before the step of drying on Büchi 190 spray-dryer, at maximum aspiration, feed: 200 ml/h (level 4), inlet T° C. 117-120° C. (level 4.5-5), outlet T° C. 78-82° C.

This provides a malt sprout fraction extract in powder form from MSF3, the powder 2204. Its characterization is detailed in table 11.

TABLE 11
Characterization of the malt sprout fraction
extract in powder form (powder 2204).

| | Powder 2204 |
|---|---|
| Dry matter dm (%) | 93.9 |
| Total nitrogen (g/kg dm) | 75.9 |
| Total FAN (g/kg dm) | 17.2 |
| Total DAN (g/kg dm) | 5.9 |
| Total PUPY | 5.4 |
| Ratio PUPY/DAN | 91.5 |

### 4.2. Effect of Malt Sprout Fraction Extract in Powder Form (Produced in Example 4.1.) in Yeast Cell Culture Media

The powdered malt sprout extract—powder 2204 (Table 11) are used as nitrogen source in yeast culture (Saccharomyces boulardii) liquid medium compared to control medium containing yeast extract (YE 0251PW-L Biospringer) as nitrogen source.

Each of the media used contained:
0.55 g N/kg,
10 g of glucose/kg of mineral elements (KH2PO4, MgSO4 7H2O and a solution of trace elements respectively 10; 5.12 and 10 g/kg).
The initial pH was adjusted to 5.5; after inoculation with the strain of S. boulardii (SuperSmart Saccharomyces boulardii—250 mg).
The 2 media (one with powder 2204 and one control with yeast extract) were incubated at 30° C. in Erlenmeyer stirring at 110 rpm.

The fermentation kinetics were followed to estimate the growth rate in the exponential phase (μ). Biomass X (yeast dry matter) produced after 24 h and 42 h of fermentation was measured after filtration (0.45μ) of samples from media, washing the filter cake and then drying at 105° C. for 24 hours. The results are presented in the following Table 12:

TABLE 12
Productions of cultures of S boulardii using different nitrogen sources: yeast extract and
malt sprout fraction extract in powder form in media at iso N concentration of 0.5 g/kg.

| | | Initial | | | | | |
|---|---|---|---|---|---|---|---|
| | Total | glucose | μ | μ | Biomass at 32 h | | Ratio to |
| Ref | nitrogen | (S0) | max | average | fermentation | X32/S0 | the control |
| extracts | g/kg | g/kg | h⁻¹ | h⁻¹ | (X32) g/kg | % | X/S |
| Control | 119.3 | 9.3 | 0.57 | 0.47 | 4.73 | 50.9 | 1 |
| YE | | | | | | | |
| Powder | 71.3 | 9.4 | 0.61 | 0.5 | 5.15 | 54.8 | 1.08 |
| 2204 | | | | | | | |

μ = Specific growth rate;
X = Biomass;
S = glucose concentration;
YE = yeast extract.

These experiments demonstrate that the malt sprout fraction extracts in powder form are excellent sources of nitrogen for the yeast culture leading to growth rates and higher biomass yields than the control medium containing yeast extract.

### 4.3. Effect of Malt Sprout Fraction Extract in Powder Form in Bacteria Culture Media

The effect of malt sprout fraction extracts in powder form in bacteria culture media have been measured and evaluated using 2 methods:
Method 1: Culture in flasks with various medium, where each nitrogen source presents in control medium (MRS and M17) such as peptone, beef extract or yeast extract is replaced by malt sprout extract extracts in powder form at the same nitrogen concentration (one by one substitution).

MRS is a selective culture medium designed to favour the luxuriant growth of Lactobacilli for lab study. Developed in 1960, this medium was named for its inventors (De Man, Rogosa and Sharpe). It contains sodium acetate, which suppresses the growth of many competing bacteria (although some other Lactobacillales, like Leuconostoc and Pediococcus, may grow). This medium has a clear brown colour.

MRS Typically Contains (Weight/Volume):
1.0% peptone
1.0% beef extract
0.4% yeast extract
2.0% glucose
0.5% sodium acetate trihydrate
0.1% polysorbate 80 (also known as Tween 80)
0.2% dipotassium hydrogen phosphate
0.2% triammonium citrate
0.02% magnesium sulfate heptahydrate
0.005% manganese sulfate tetrahydrate
pH adjusted to 6.2 at 25° C.

The yeast and meat extracts and peptone provide sources of carbon, nitrogen and vitamins for general bacterial growth. The yeast extract also contains vitamins and amino acids specifically required by Lactobacilli. Polysorbate 80 is a surfactant which assists in nutrient uptake by Lactobacilli. Magnesium sulfate and manganese sulfate provide cations used in metabolism.

M17 is a selective media recommended as an improved medium for the growth and enumeration of lactic streptococci and their bacteriophages

M17 Typically Contains:

Approximate Formula (per 950 mL):
5.0 g of Pancreatic Digest of Casein
5.0 g of Soy Peptone
5.0 g of Beef Extract
2.5 g of Yeast Extract
0.5 g of Ascorbic Acid
0.25 g of Magnesium Sulfate
19.0 g of Disodium-β-glycerophosphate
Method 2: Culture on agar plates with malt sprout fraction extract in powder form as only source of nitrogen (total substitution).

Medium Preparation:
preparation of a stock solution of each nitrogen sources,
preparation of a stock solution of each salt solutions,
preparation of a stock solution of each sugar solutions,
mix of several elements according to the needs,
pH adjustment to 6.5 for medium MRS and pH to 7.2 for medium M17,
centrifugation to eliminate the turbidity (this turbidity is related to addition of RDM extract),
the media is sterilized by filtration and transferred in flasks previously sterilized by autoclaving.

Seed Preparation:
the seed culture is grown within the commercial media,
at the end of growth the seeds are centrifuged, the pellets are collected in the physiological water and are centrifuged again,
the pellets are new collected in the physiological water and are distributed in the flasks.

This protocol avoids the intake of undesired nutrients in the medium. The size of inoculums was defined by previous tests.

Culture in Flask
The culture conditions are: static growth at 37° C.

The growing is monitored by:
OD 600 nm,
pH,
[glucose] (YSI) for Lactobacillus,
[lactose] (HPLC) for Lactococcus,
[Organics acid], [ethanol] (HPLC),
Dry Matter after filtration on the final time,
Microbiological enumeration on plat count agar with MRS and M17 media

### Culture on Plates Agar

the culture conditions are: static growth at 37° C. under controlled atmosphere via a candle jar,
three types of medium are tested,
   commercial medium MRS or M17,
   simulated commercial medium with commercial nitrogen sources,
   Malt sprout fraction extract in powder trials (substitution of every other nitrogen sources by MSFE in powder),
for the inoculation, 5 spots of 0.1 μl of the different cultures are deposited on the surface of each plates,
the growing of the spot is measured each days during 3 days.

The malt sprout fraction extract in powder used in these experiments corresponds to the powder 2204 (characterized on table 11).

### 4.3.1. Lactobacillus Strains

The cultures of Lactobacillus bacteria in liquid media were studied taking as positive control the medium Man Rogosa Sharpe (MRS) from AES (ref AEB140652). The different sources of nitrogen of the medium MRS are Casein peptones (ref A1402HA), meat extract (ref A17HA) and yeast extract (ref A1202HA) which are available from Biokar.

These cultures have been prepared according to methods 1 and 2 described above.

The following strains were used:
Lactobacillus acidophilus (ATCC(=American type culture collection) 4356)
Lactobacillus delbrueckii subsp bulgaricus (ATCC11 842).

The results are presented in the following Table 13:

TABLE 13
Results of cultures of two lactobacillus strains in media
containing malt sprout fraction extract in powder (powder 2204)

| | AES | | | Powder | Powder | Powder |
|---|---|---|---|---|---|---|
| | Control | [symbol] AES [symbol] | Powder | 2204 in | 2204 in | 2204 in |
| | (ref | Control | 2204 | substitution | substitution | substitution |
| Media | AEB140652) | (recomposed) | 100% | YE | PP | EV |
| Powder | 0 | 0 | 49.79 | 8.21 | 22.83 | 18.75 |
| 2204 | | | | | | |
| MS g/L | | | | | | |
| N g/L | 3.28 | 3.59 | 3.45 | 3.61 | 3.52 | 3.57 |

Lactobacillus acidophilus

| | | | | | | |
|---|---|---|---|---|---|---|
| μ (h⁻¹) | 0.15 | 0.2 | 0.44 | 0.45 | 0.47 | 0.48 |
| 10⁹ | 0.13 | 0.94 | 1.8 | 1.8 | 2.5 | 2.2 |
| UFC/mL | | | | | | |
| Lactic | 12.3 | 17.8 | 23 | 21.7 | 22.1 | 21.7 |
| acid g/L | | | | | | |

Lactobacillus delbrueckii

| | | | | | | |
|---|---|---|---|---|---|---|
| μ (h⁻¹) | 0.032 | 0.048 | 0.595 | 0.394 | 0.62 | 0.595 |
| 10⁸ | 0.77 | 0.43 | 5.75 | 3.5 | 6.9 | 4.5 |
| UFC/mL | | | | | | |
| Lactic | 9.6 | 5.6 | 8.7 | 10.9 | 10.3 | 9.9 |
| acid g/L | | | | | | |

Powder 2204 is used in the formulation of AES media recomposed from nitrogen sources from Biokar in substitution of one of these nitrogen sources or all 3 (EFM 100%).
YE: yeast extract
PP: polypeptones of casein
EV: meat extract

### 4.3.2. Lactococcus and Streptococcus Strains

The following bacteria were investigated:
Lactoccus lactis subsp lactis (ATCC 11454)
Streptococcus salivarius subsp thermophilus (ATCC19 258).

Taking as control the M17 medium Sigma Aldrich (ref. 56156 M17 broth). Tryptone (ref A1401HA), meat peptones (ref A1708HA) and soy (ref A1601HA), meat extract (ref A170HA) and yeast extract (ref A1202HA) are available from Biokar. The lactose concentration was increased to 20 g/L. The culture conditions and the measurements are similar to that of the preceding example. The results are presented in the following Table 14:

TABLE 14
Results of cultures of two strains lactococcus and streptococcus in media containing malt
sprout fraction extract in powder.

| | | | | Powder | Powder | Powder | Powder | Powder |
|---|---|---|---|---|---|---|---|---|
| | SIGMA | [symbol] SIGMA [symbol] | Powder | 2204 in | 2204 in | 2204 in | 2204 in | 2204 in |
| | Commercial | Recomposed | 2204 | substitution | substitution | substitution | substitution | substitution |
| Media | Control | Control | 100% | YE | PS | EV | T | PV |
| Powder | 0 | 0 | 31.04 | 4.11 | 7.65 | 9.37 | 4.81 | 5.11 |
| 2204 | | | | | | | | |
| Dry | | | | | | | | |
| Matter | | | | | | | | |
| g/L | | | | | | | | |
| N g/L | 2.0 | 2.09 | 2.05 | 2.1 | 2.05 | 2.1 | 2.1 | 2.06 |

Lactococcus lactis

| | | | | | | | | |
|---|---|---|---|---|---|---|---|---|
| μ (h⁻¹) | 1.2 | 0.71 | 1.26 | 1 | 1.18 | 1.23 | 0.822 | 1.09 |
| Lactic | 8.31 | 7.5 | 9.2 | 8.7 | 8.8 | 8.9 | 8.69 | 8.7 |
| acid g/L | | | | | | | | |

Streptococcus salivarius

| | | | | | | | | |
|---|---|---|---|---|---|---|---|---|
| μ (h⁻¹) | 1.03 | 0.9 | 0.99 | 0.73 | 0.95 | 1.11 | 0.88 | 0.97 |
| 10⁹ | nd | 1.1 | nd | 1.5 | 4.4 | 2.4 | 2.9 | 7.1 |
| UFC/mL | | | | | | | | |
| Lactic | 7.57 | 8.35 | 8.56 | 8.58 | 8.71 | 8.8 | 8.55 | 8.7 |
| acid g/L | | | | | | | | |

Powder 2204 is used in the formulation media SIGMA recomposed from nitrogen sources from Biokar in substitution of one of these nitrogen sources or all 5 (EFM 100%).
YE: yeast extract
PS: Soy peptone
EV: meat extract
PV: Meat peptone
T: Tryptone
(*): Counts higher than 10¹⁰

We further proceed to the culture of four strains disclosed in examples 4.3.1 and 4.3.2 in solid agar media.

These cultures were performed on agar plates, static at 37° C. under controlled atmosphere Candle jars. Three types of media were used:
MRS or M17 media (same references than in examples 4.3.1 and 4.3.2),
MRS and M17 media recomposed from commercial sources of nitrogen,
media consisting of malt sprout fraction extract in powder (PMSFE) in substitution of the total nitrogen sources
   5 drops of 0.1 μl of different cultures are deposited on the surface of each agar media box. Growth is measured each day for 3 days.

The results are presented in the following Table 15:

TABLE 15
Culture of lactic acid bacteria strains in agar media -
comparison of colony diameters (in millimeters mm).

| | 24 h | 48 h | 72 h |
|---|---|---|---|
| L acidophilus | | | |
| MRS | 5.6 | 6.1 | 6.4 |
| MRS recomposed | 6.3 | 6.8 | 7.3 |
| PMSFE medium | 7.3 | 8.1 | 8.4 |
| L delbrueckii | | | |
| MRS | 5.2 | 6.6 | 7.3 |
| MRS recomposed | 7.3 | 8 | 9.1 |
| PMSFE medium | 7.2 | 8.3 | 8.7 |
| Lactococcus lactis | | | |
| M17 | 4.9 | 5.6 | 6 |
| PMSFE medium | 4.6 | 5 | 5.6 |

The tested strains can grow on media exclusively made of malt sprouts extracts. One can therefore consider total or partial substitution of conventional sources of nitrogen by malt sprout extracts.

### Additional Results

The result obtained with PMSFE on the growth for three various strains of lactic acid bacteria are good. Each strains tested are able to use the PMSFE as a substitute product to replace the nitrogen source and also may be a part of the sugar source.

With Lactococcus acidophilus the PMSFE brings something which extends the growth and the results prove that PMSFE extract seems to be well adapted for the growth. Moreover the lactic acid concentrations seem to be highly correlated to the total carbon concentration and in our case with the use of PMSFE extract.

The using of PMSFE brings a high benefit to the growth of Lactobacillus delbrueckii subsp bulgaricus (every trials with PMSFE are significantly better than the controls) but also for the viability of this strain.

However, the Lactobacillus strain especially Lactobacillus acidophilus requires compound in yeast extract for his growth. A partial substitution may be considered.

### Example 5: Preparation of a Liquid Malt Sprout Fraction Extract from Optimized Diffusion Process

Malt sprout fraction used to produce this liquid malt sprout fraction extract is characterized in tables 5 and 6 (MSFE2)

An aqueous extract from MSFE2 is prepared according to the following process. A malt sprout fraction suspension and osmotic water in the weight ratio of 1/10 in order to make diffusion. The said diffusion was carried out at 20° C., during 2 hours. At the end of the 2 hours, a finishing step is carried out as follows: pH adjustment is realized at pH=8, and the aqueous extract is heated at 100° C. for 20 minutes.

Then a first liquid-solid separation step is realized on aqueous extract, by centrifugation (RC12BP+at 13000 g, 30 min, 20° C.) in order to remove all insoluble particles.

Characterization of clarified and final aqueous extract is detailed in table 16.

TABLE 16
Characterization of liquid malt sprout fraction
extract (MSFE2L) obtained from optimized diffusion process:

| | MSFE2L (Optimized |
|---|---|
| | diffusion process) |
| Dry matter dm (%) | 1.5 |
| Total nitrogen (g/kg dm) | 50.6 |
| Total FAN (g/kg dm) | 15.70 |
| Total DAN (g/kg dm) | 13 |
| Total PUPY (g/kg dm) | 10.86 |
| Ratio PUPY/DAN | 83.5 |

### Example 6: Effect of Malt Sprout Fraction Extract in Syrup Form (Also Named Syrup or Slurry Concentrate and MSFE2) in Lactic Acid Bacteria Culture Media

The MSFE2 as prepared in example 2 and characterized in table 8 was used in the following test. These cultures were performed on agar plates, static at 37° C. under controlled atmosphere Candle jars. Three types of media were used:
MRS (same references than in examples 4.3.1 and 4.3.2),
MRS media recomposed from commercial sources of nitrogen,
media consisting of syrup in substitution of the total nitrogen sources EFM
   5 drops of 0.1 μl of different cultures are deposited on the surface of each agar media box. Growth is measured each day for 3 days.

TABLE 17
Culture of L.acidophilus vs MRS
media on agar media - y comparison of
colony diameter (in millimeters mm).

| L acidophilus | 18 h | 24 h | 42 h |
|---|---|---|---|
| MRS | 7.06 | 7.07 | 7.24 |
| MRS recomposed | 6.88 | 7.10 | 7.3 |
| EFM medium | 6.31 | 6.32 | 6.48 |

The tested strain can grow also on media exclusively made of malt sprouts extracts in powder form (Example 4, Table 17), and in liquid concentrated form (example described here). One can therefore consider total or partial substitution of conventional sources of nitrogen by malt sprout extracts.

### Example 7: Interest of the Finishing Step in Combination with Diffusion or Hydrolysis

Malt Sprout Extracts Preparation (MSFE 2, 3, 4):
a) Mixing of 1 part of malt sprout fraction with 10 parts of water (=slurry) in a tank with stirring device.
b) Diffusion of the solubles into water during 2 h hours at ambient temperature (20° C.) OR diffusion of the solubles into water during 2 h hours at 50° C. OR hydrolysis with Sumizyme LP and Protease Plus (at 0.3% of malt sprout fraction dry matter).
c) After 2 or 5 hours, apply or not he following heat treatment: adjustment of the pH slurry at 8 and heating up to boiling point (100° C.) during 5 min.
d) The slurry is then centrifuged at 13000 g during 10 min.
e) The supernatant is then recovered and constitutes the malt sprout fraction extract.

| Ref | MSFE 2 | MSFE 3 | MSFE 4 |
|---|---|---|---|
| Process used to | Diffusion at | Hydrolysis at | Hydrolysis at |
| prepare MSFE | 20° C.-2 h | 5 h-50° C. | 5 h-50° C. |
| Malt sprouts | 18 | 29 | 29 |
| fractions protein | | | |
| content % | | | |
| N % Malt sprouts | 2.97 | 4.64 | 4.64 |
| Finishing step | pH 8 - 100° C.- | No | pH 8 - 100° C.- |
| | 20 mn | | 20 mn |
| DM in extract % WV | 1.47 | 4.2 | 4.65 |
| DAN mg/L extract | 177 | 399 | 379 |
| PUPY mg/L extract | 92.45 | 355 | 317 |
| PUPY/DAN | 0.52 | 0.89 | 0.84 |
| Nt g/L extract | 0.7 | 2.9 | 3 |
| FAN mg/L extract | 141* | 702 | 685 |

*Estimated
OBSERVATIONS/CONCLUSIONS: In those experiments we searched to find extractions conditions giving concomitantly high concentrations of soluble extracts (DM in extract %) and DAN, and high ratios of PUPY/DAN. Nt (total nitrogen in the extracts) and FAN are given as indicators of N and organic N compounds enrichment.

With selected malt sprout fraction (29% crude protein) hydrolysis alone and hydrolysis with finishing treatment allow to obtain concomitantly high concentrations of soluble extracts (respectively 4.2 and 4.65%, high concentrations of DAN (respectively 399 and 379 mg/L) and PUPY (355 and 317 mg/L) and PUPY/DAN ratio above 80% (respectively 0.89 and 0.84). However, DAN, PUPY and FAN concentrations in MSFE 4 are lower than in MSFE 3 although DM in extract is higher. It is an indication that 20 mn heat treatment is too severe after 5 h hydrolysis at 50° C. and result in losses of organic nitrogen compounds. Heat treatment conditions should be further optimized in relation with hydrolysis conditions to fine tune the best extraction processes.

These experiments show that it is possible to obtain extensive hydrolysis of nucleic acids and high concentrations of soluble extracts and that further optimization is feasible.

### Example 8: Growth of Lactic Acid Bacteria in Model Fermentation Media Demonstrate the Effect of PUPY Supplementation on Bacterial Growth

1. Fermentation Media Ingredient Characterization

| | | | Total | FAN | DAN | PUPY |
|---|---|---|---|---|---|---|
| | | Supplier/ | nitrogen (N) | g/kg | g/kg | g/kg |
| Ingredient | Used as | product code | g/kg dm | dm | dm | dm |
| Soy | Source of | Solabia/ | 98.9 | 24.73 | 1.65 | 0.37 |
| peptone | nitrogen | Soy peptone | | | | |
| | | F-A1603 | | | | |

The pure purines and pyrimidines used in this experiment were supplied by Sigma-Aldrich under the following product references: A8626, C3506, U0750, G11950, H9377. These products have a purity >99%.

2. Fermentation Parameters

Equipment:
Microplate reader TECAN Infinite 200 Pro, connected to computer using Magellan or Icontrol software.
96 wells microplate

Measured Parameter:

Optical density (OD) at 600 nm wavelength—To measure bacterial concentration.

Fermentation Kinetics Parameters:
12 h pre-culture in MRS broth (from DIFCO)
Total volume/well: 200 μL
Percent inoculum addition: 10%
Number of replicates=3
Stirring: 3 mm of orbital amplitude during 15 minutes
Temperature=30 or 37° C. depending on the strain
Follow-up over 24 hours with measurement every 30 minutes Strains
Lactobacillus helveticus LAFTI L10 (cultivated at 37° C.)
Lactobacillus plantarum ATCC 14917 (cultivated at 30° C.)
Pediococcus pentosaceus ATCC 33316 (cultivated at 30° C.)

3. Fermentation Media Formulation

Three Basic Solutions were Prepared in Order to Produce 4 Media:

| | PUPY concentration | FAN concentration |
|---|---|---|
| Media | (mg/L) | (mg/L) |
| 1 | 0 | 390 |
| 2 | 0 | 780 |
| 3 | 150 | 390 |
| 4 | 150 | 780 |

A base medium composed of glucose and mineral salts: 500 mL at 120.6 g/L (M)

| | Concentration | Mass for 500 mL |
|---|---|---|
| Ingredients | (g/L) | solution (g) |
| Glucose | 80 | 40 |
| Tween 80 | 4 | 2 |
| Dipotassium phosphate K2HPO4 | 8 | 4 |
| Sodium acetate C2H3NaO2 | 20 | 10 |
| Ammonium citrate C6H5 + 4yFexNyO7 | 8 | 4 |
| Magnesium sulfate MgSO4 | 0.4 | 0.2 |
| Manganese sulfate MnSO4 | 0.2 | 0.1 |

A nitrogen solution composed of soy peptone: 300 mL at 60 g/L (N)

| | Concentration | Mass for |
|---|---|---|
| Ingredient | (g/L) | 300 mL (g) |
| Soy peptone | 60 | 18 |

A PUPY solution composed of pure purines and pyrimidines bases: 800 mL at 3 g/L (PUPY)

| | | Concentration | Mass for 800 |
|---|---|---|---|
| PUPY | % | (g/L) | mL (g) |
| C | 3 | 0.09 | 0.0720 |
| H | 55 | 1.65 | 1.3200 |
| U | 25 | 0.75 | 0.6000 |
| G | 8 | 0.24 | 0.1920 |
| A | 9 | 0.27 | 0.2160 |
| Total | 100 | 3.00 | |

Final fermentation medium formulation:

| Media | Volume (M) | Volume (PUPY) | Volume (N) | Volume (water) | Total volume |
|---|---|---|---|---|---|
| 1 | 25 mL | 0 mL | 25 mL | 50 mL | 100 mL |
| 2 | 25 mL | 0 mL | 50 mL | 25 mL | 100 mL |
| 4 | 25 mL | 5 mL | 25 mL | 45 mL | 100 mL |
| 3 | 25 mL | 5 mL | 50 mL | 20 mL | 100 mL |

### 4. Results

| | Maximum Absorbance (OD at 600 nm) | | | |
|---|---|---|---|---|
| | PUPY 0 mg/L | | PUPY 150 mg/L | |
| Strains | FAN 390 | FAN 780 | FAN 390 | FAN 780 |
| | mg/L | mg/L | mg/L | mg/L |
| L. plantarum ATCC 14917 | 1.367 | 1.506 | 1.608 | 1.578 |
| P. pentosaceus ATCC 33316 | 1.425 | 1.426 | 1.563 | 1.531 |
| L. helveticus LAFTI L10 | 1.357 | 1.550 | 1.547 | 1.436 |

In the non-supplemented media (PUPY=0) the bacteria can find available FAN to support their growth and the maximum concentration of biomass is obtained at the highest concentration of FAN.

In the supplemented media (PUPY=150), the bacteria can find the available FAN to support their growth, but the maximum concentration of biomass (Amax) is obtained at the lowest concentration of FAN (390 mg/L). The maximum concentrations of biomass are obtained at FAN=390 mg/L and DAN=150 mg/L. However, for L helveticus, Amax is practically equal to Amax obtained at FAN 780-DAN 0, and the highest Amax are obtained for L plantarum and P pentosaceus at FAN390-DAN 150. This unexpected result is particularly interesting because it shows complementarity and or synergy between FAN and DAN.

### Example 9: The Use of Malt Sprout Fraction Extract as Substitute to Conventional Nitrogen Sources in Media Formulation for Lactic Acid Bacteria

The standard media to grow lactic acid bacteria (of genus Lactobacilli) is MRS broth. This media is constituted of various nitrogen sources: proteose-pepton, Meat extract, and yeast extract.

In these experiments, MSFE is used as substitute of all nitrogen sources (w/w) and of each of the nitrogen sources.

1. Characterization of Nitrogen Sources

| | | FAN | FAN | DAN | | PUPY | | | |
|---|---|---|---|---|---|---|---|---|---|
| | | g/kg | (g/L) | g/kg | DAN | g/kg | PUPY | | |
| | Quantity | dm in | in | dm in | (g/L) in | dm in | (g/L) in | | |
| N | (g) in 55 | N | media | N | media | N | media | | |
| sources | g dm | source | 55 g/L | source | 55 g/L | source | 55 g/L | DAN/FAN | PUPY/FAN |
| 1 | 10 | 12.24 | 0.1224 | 4.62 | 0.0462 | 2.05 | 0.0205 | 0.377 | 0.1675 |
| Proteose- | | | | | | | | | |
| pepton | | | | | | | | | |
| 2 Beef | 10 | 19.96 | 0.1996 | 2.44 | 0.0244 | 0.911 | 0.00911 | 0.1222 | 0.0456 |
| meat | | | | | | | | | |
| extract | | | | | | | | | |
| 3 Yeast | 5 | 36.1 | 0.1805 | 7.93 | 0.03965 | 1.6 | 0.008 | 0.2197 | 0.0443 |
| extract | | | | | | | | | |
| MSFE | | 10.58 | | 5.965 | | 5.387 | | 0.564 | 0.509 |
| 022204 | | | | | | | | | |

2. Media Formulations

| | | FAN | FAN | DAN | | PUPY | | | |
|---|---|---|---|---|---|---|---|---|---|
| | | g/kg | (g/L) | g/kg | DAN | g/kg | PUPY | | |
| | Quantity | dm in | in | dm in | (g/L) in | dm in | (g/L) in | | |
| N | (g) in 55 | N | media | N | media | N | media | | |
| sources | g dm | source | 55 g/L | source | 55 g/L | source | 55 g/L | DAN/FAN | PUPY/FAN |
| Medium 1 | 10 | 10.58 | 0.1058 | 5.965 | 0.05965 | 5.387 | 0.05387 | | |
| MSE | | | | | | | | | |
| substitutes | | | | | | | | | |
| Proteose | | | | | | | | | |
| pepton | | | | | | | | | |
| 2 + 3 | 15 | | 0.3801 | | 0.06405 | | 0.01711 | | |
| Medium 1 | 55 | | 0.4859 | | 0.1237 | | 0.07098 | 0.255 | 0.146 |
| Medium 2 | 10 | 10.58 | 0.1058 | 5.965 | 0.05965 | 5.387 | 0.05387 | | |
| MSE | | | | | | | | | |
| substitutes | | | | | | | | | |
| meat | | | | | | | | | |
| extract | | | | | | | | | |
| 1 + 3 | 15 | | 0.3029 | | 0.08585 | | 0.0285 | | |
| Medium 2 | 55 | | 0.4087 | | 0.1455 | | 0.08237 | 0.356 | 0.202 |
| Medium 3 | 5 | 10.58 | 0.0529 | 5.965 | 0.029825 | 5.387 | 0.026935 | | |
| MSE | | | | | | | | | |
| substitutes | | | | | | | | | |
| yeast | | | | | | | | | |
| extract | | | | | | | | | |
| 1 + 2 | 20 | | 0.322 | | 0.0706 | | 0.02961 | | |
| Medium 3 | 55 | | 0.3749 | | 0.100425 | | 0.056545 | 0.296 | 0.151 |
| Control | 25 | | 0.5025 | | 0.11025 | | 0.03761 | 0.219 | 0.075 |
| medium | | | | | | | | | |
| No | | | | | | | | | |
| substitution | | | | | | | | | |
| 1 + 2 + 3 | | | | | | | | | |

3. Impact of the Substitution of Conventional Nitrogen Sources on the Maximum Concentration (Amax) of Different Lactic Acid Bacteria

| | A max in | | A max in | |
|---|---|---|---|---|
| | CONTROL | SUBSTITUTED | SUBSTITUTED | Improvement |
| Strains | MEDIUM | MEDIUM | MEDIUM | factor |
| Lactobacillus | 0.452 | Medium 2 | 0.922 | 2.04 |
| acidophilus | | | | |
| ATCC 4356 | | | | |
| Lactobacillus | 0.449 | Medium 3 | 0.501 | 1.12 |
| delbrueckii lactis | | | | |
| ATCC 1215 | | | | |
| Lactobacillus | 1.482 | Medium 1 | 1.594 | 1.08 |
| helveticus | | | | |
| LAFTI L10 | | | | |

Depending on the N compounds requirements of the strains, MSE can substitute N sources like proteose-pepton, meat extract or yeast extract with improved final biomass concentration. It can be observed that all the substituted media have a lower concentration of FAN than the control medium but higher PUPY concentration and PUPY/FAN ratio. These very surprising results show that MSE offer the unique possibility to reduce FAN requirements when proper levels of DAN and PUPY are present in the media. It is particularly interesting to optimize performances of fermentations.

### Example 10: The Use of Malt Sprout Fraction Extract as Flavor Enhancer in Chicken Broth

1. Characterization of Malt Sprout Extract Used in the Experiment.

| | | | Dry | Total | FAN | DAN | PUPY | Ratio |
|---|---|---|---|---|---|---|---|---|
| | | | matter | nitrogen | g/kg | g/kg | g/kg | PUPY/DAN |
| | Used as | Form | % | (N) g/kg dm | dm | dm | dm | % |
| MSFE | Flavor | Powder | 95 | 66.4 | 9.71 | 6.67 | 6.41 | 96.1 |
| ORG2205 | enhancer | | | | | | | |

2. Basic Matrix: Homemade Chicken Broth

For the production of about 1 liter of chicken broth

Ingredients:
1 chicken carcass (cut into pieces)—for an average chicken about 300 g.
1 carrot—approx. 100-120 g
1 celery stalk—approx. 40 g
1 white leek—approx. 120 g
1 yellow onion—approx. 100 g
1 branch of thyme—2 g
1 branch of laurel—4-5 medium leaves
1 pinch of salt—3 g
1.2 liters of water

Recipe:

1. Peel and cut into coarse pieces carrot, celery stalk and leek.

2. Cut the onion into 4.

3. In a casserole, fry the pieces of chicken carcass until lightly browned.

4. Add the carrot, celery and leek, as well as the onion.

5. Cover with water

6. Add the pinch of salt, thyme and bay leaf.

7. Simmer without cover for about 1 hour without boiling.

8. When cooking is complete, let everything cool in the pan.

9. Once the mixture has cooled, filter with a strainer to recover the broth.

10. Mix the different productions to obtain a single batch.

11. Store covered broth in refrigerator (½ day).

12. Before freezing the juice, remove the layer of fat that is on the surface (degreasing).

4 concentrations of the malt sprout fraction extract have been tested: 0% 0.5% 0.8% 1% (g/100 g of chicken broth).

3. Sensory Profile Methodology
a) Methodology: Sensory profile
   NF EN ISO 13299 May 2010 (Sensory analysis-Methodology-General guidelines for the establishment of a sensory profile)
   NF EN ISO 8586 February 2014 (Sensory analysis-General guidelines for the selection, training and control of qualified subjects and expert sensory subjects)
b) Protocol
   15 people were recruited from the external panels trained by SensoStat.
   5 training sessions of approximately 1 hour were conducted over 1 month. The panel was therefore qualified to analyze the type of product studied.
   The subjects were trained on a list of descriptors determined in agreement with Malteurop and with their feelings during the sessions, using classifications and identification of flavors and aromas, scale tests, presentation of references, group discussions on different descriptors. The products used in this phase were purchased commercially or consisted of “homemade” chicken broth with different concentrations of the test compound.
   The study took place in the premises of the Center for Taste and Food Sciences (Dijon), in individual tasting booths, under standardized light conditions and temperatures.
   The cabins are computerized to automate data acquisition (FIZZ).
   The cabins were lit up in white light.
   The training sessions took place in individual cabins or in a meeting room when a discussion was needed.
c) Measurement sessions
   The measurement sessions took place on Dec. 1, 2017. The 4 concentrations were evaluated on the list of 6 descriptors:
      Vegetables
      Chicken
      Umami
      Salt
      Aromatic herbs
      Fat
   The products were evaluated on continuous scales from 0 to 10
   The evaluation of the 4 recipes was made with the monadic order of presentation following a Latin square, with a repetition (each recipe was evaluated twice by each subject).
   The products were served blind, in black cups (to avoid visual differences) coded (about 20 ml), at about 45° C.
   A 2-minute break was observed between each sample. Rinsing was done with water and unsalted crackers to optimize mouth cleaning.

4. Results
a) Analyse of variance: product effect

| | | | | | P value |
|---|---|---|---|---|---|
| Products: | 0% | 0.5% | 0.8% | 1% | Product effect |
| Vegetable | 7.53 | 7.603 | 7.547 | 7.107 | 0.579 |
| Chicken | 7.363 a | 7.337 ab | 6.643 bc | 6.467 c | 0.082 * |
| Umami | 6.477 b | 6.587 b | 7.267 a | 7.353 a | 0.064 * |
| Salt | 6.333 b | 6.21 b | 7.1 a | 6.733 ab | 0.042 ** |
| Aromatic herbs | 5.637 | 5.69 | 6.23 | 6.313 | 0.319 |
| Fat | 1.653 b | 1.527 b | 2.217 a | 1.907 ab | 0.080 * |

Anova model: subject+product

Fisher test (LSD)/Analysis of differences between products with a 90% confidence interval

Products linked by the same letter are not statistically different
b) Observations:
   0.8% and 1% samples:
      Higher intensity on Umami, Salty and Fat (statistically significant differences)
      Lower Intensity on Chicken (statistically significant difference)
   No effect of the test compound on the descriptors Vegetables and Aromatic herbs.
c) Conclusions

The 0.8% and 1% samples have a higher intensity than the 0.5% and 0% products on the Umami, Salty and Fat descriptors. They have a lower intensity on the Chicken descriptor.

No effect of the test compound was demonstrated on the Vegetable and Aromatic herb descriptors.

We can Therefore Conclude that at 0.8% and 1%, the Studied Compound has an Enhancing Effect on Umami and Salty, and on a Fat “Taste”.

### Example 11: Comparative Composition of Standard Malt Sprout Extract and Malt Sprout Fraction Extracts Prepared According to the Invention

Preparation of Malt Sprout Extract (MSE) and Malt Sprout Fraction Extract (MSFE) and Characterization of the Compositions of the Resulting Extracts.

Different methods or processes including raw material selection to prepare malt sprout extracts are described in some published documents. Very limited information is available on the characteristics of the resulting extracts. The compositions are poorly defined. The in-depth research works that the inventors have carried out on MSFE demonstrate the importance of nucleic acid hydrolysis to obtain defined DAN and PUPY concentrations in MSFE and a defined ratio of PUPY/DAN.

Nothing is published in the prior art on the concentrations of DAN and PUPY in malt sprouts extracts obtained with the different existing methods or processes. These methods or processes including raw material selection differ from one to another but the absence of knowledge on DAN and PUPY concentrations, or other relevant components, in the respective extracts does not allow to differentiate the composition of these extracts.

1. Preparation of Standard Malt Sprout Extract (MSE) and Malt Sprout Fractions Extracts (MSFE)

For this experiment, a malt sprout extract (MSE) was prepared according to a conventional method using common malt sprouts (18-23% crude proteins) from a usual malting process. The resulting extract (MSE c) was used as a control to define its composition.

Two malt sprout fraction extracts (MSFE) were prepared from a selected malt sprout fraction (MSF4) obtained from the same common malt sprout. The selected malt sprout fraction contained more than 80% rootlets and acrospires (and between 28 and 34% crude proteins).

One malt sprout fraction extract (MSFE4-A) was obtained using a diffusion method followed by a finishing step. A second malt sprout fraction extract (MSFE4-B) was obtained using a hydrolysis method in the presence of exogenous enzymes, followed by a finishing step.

Both MSFE4-A and MSFE4-B, obtained according to the claimed methods of preparation, were analyzed to define the compositions and compare with MSE c.

The remaining solid residues resulting from the 3 methods of preparation were also recovered and analyzed to determine the compositions and to establish the respective overall mass balance and Nitrogen components mass balances.

2. Comparative Analytical Characterizations of MSE and MSFE

The analytical work included:

Analysis of common malt sprout and selected malt sprout fraction including: dry matter, extract, Nt, crude protein, soluble protein, FAN, diastasic power, nucleic acids, DAN, PUPY, pentoses, crude fibers, soluble and insoluble fibers, soluble β-glucans, starch, total sugars, fats, ashes.

Analysis of extracts MSE c, MSFE4-A and MSFE4-B including: soluble dry matter, Nt, crude protein, soluble protein, FAN, amino acids, diastasic power, oligonucleotides, DAN, PUPY, pentoses (xylose, arabinose, ribose, deoxyribose), glucose, crude fibers, soluble and insoluble fibers, soluble β-glucans, starch, total sugars, fats, ashes.

Analysis of remaining solid residues including: wet mass after rinsing, dry matter, Nt, crude protein, soluble protein, FAN, diastasic power, nucleic acids, DAN, PUPY, pentoses, crude fibers, soluble and insoluble fibers, soluble β-glucans, starch, total sugars, fats, ashes.

Compositions of the 3 extracts are compared to establish critical difference in selected components and the advantages of the claimed extracts and methods.

1-29. (canceled)

30. Composition containing malt sprouts fraction extract, wherein the composition comprises at least 0.5%, and less than 98% of dry matter by total weight of the composition, and comprising:
total Nitrogen content from 40 to 140 g/kg of dry matter
free Amino Nitrogen content from 6 to 60 g/kg of dry matter, and
nucleic Acid Derivatives such as purines and pyrimidines bases in an amount from 3 to 25 g/kg of dry matter,
PUPY content from 1 to 20 g/kg of dry matter,
ratio PUPY/Nucleic Acid Derivatives higher than 20%.

31. Composition according to claim 30, consisting of malt sprouts fraction extract wherein the malt sprouts fraction extract comprises at least 0.5%, and less than 98% of dry matter by total weight of the malt sprouts fraction extract and comprising:
total Nitrogen content from 40 to 80 g/kg of dry matter
free Amino Nitrogen content from 6 to 30 g/kg of dry matter, and
nucleic Acid Derivatives such as purines and pyrimidines bases in an amount from 4 to 25 g/kg of dry matter,
PUPY content from 3 to 20 g/kg of dry matter,
ratio PUPY/Nucleic Acid Derivatives higher than 80%.

32. Composition according to claim 30, consisting of malt sprouts fraction extract wherein the extract is in a liquid form comprising at least 0.5%, and less than 1.1%, or at least 1.1%, and less than 3.5%, or at least 3.5%, and less than 5%, or at least 5%, and less than 7% of dry matter by total weight of the malt sprouts fraction extract and comprising:
total Nitrogen content from 40 to 80 g/kg of dry matter
free Amino Nitrogen content from 6 to 30 g/kg of dry matter, and
nucleic Acid Derivatives such as purines and pyrimidines bases in an amount from 5 to 25 g/kg of dry matter,
PUPY content from 4 to 20 g/kg of dry matter,
ratio PUPY/Nucleic Acid Derivatives higher than 80%.

33. Composition according to claim 30, consisting of malt sprouts fraction extract wherein the extract is in a liquid form, or a concentrated juice, comprising at least 7%, and less than 10%, or at least 10%, and less than 15% of dry matter by total weight of the malt sprouts fraction extract and comprising:
total Nitrogen content from 40 to 80 g/kg of dry matter
free Amino Nitrogen content from 6 to 30 g/kg of dry matter, and
nucleic Acid Derivatives such as purines and pyrimidines bases in an amount from 4 to 25 g/kg of dry matter,
PUPY content from 3 to 20 g/kg of dry matter,
ratio PUPY/Nucleic Acid Derivatives higher than 80%.

34. Composition according to claim 30, consisting of malt sprouts fraction extract wherein the extract is in a liquid form or a solid form, or a concentrated juice, a slurry or a paste, comprising at least 15%, and less than 60% of dry matter by total weight of the malt sprouts fraction extract and comprising:
total Nitrogen content from 40 to 80 g/kg of dry matter
free Amino Nitrogen content from 6 to 30 g/kg of dry matter, and
nucleic Acid Derivatives such as purines and pyrimidines bases in an amount from 4 to 25 g/kg of dry matter,
PUPY content from 3 to 20 g/kg of dry matter,
ratio PUPY/Nucleic Acid Derivatives higher than 80%.

35. Composition according to claim 30, consisting of malt sprouts fraction extract wherein the extract is in a solid form, or a paste or a powder, comprising at least 60%, and less than 98% of dry matter by total weight of the malt sprouts fraction extract and comprising:
total Nitrogen content from 40 to 80 g/kg of dry matter
free Amino Nitrogen content from 6 to 30 g/kg of dry matter, and
nucleic Acid Derivatives such as purines and pyrimidines bases in an amount from 4 to 25 g/kg of dry matter,
PUPY content from 3 to 20 g/kg of dry matter,
ratio PUPY/Nucleic Acid Derivatives higher than 80%.

36. Composition consisting of malt sprouts fraction extract according to of claim 30, wherein the malt is selected from barley, wheat, rye, spelt, corn, millet sorghum, oat, triticale, rice and mixtures thereof, and obtained from an aqueous extraction of malt sprouts fractions obtained by a sieving process and comprising a content of rootlets and acrospires higher than 50%, or higher than 60%, or higher than 65%, or between 65% and 85% by total weight of fractions.

37. Composition consisting of malt sprouts fraction extract, wherein the composition comprises at least 0.5%, and less than 98% of dry matter by total weight of the composition, and comprising:
total Nitrogen content from 40 to 140 g/kg of dry matter
free Amino Nitrogen content from 6 to 60 g/kg of dry matter, and
nucleic Acid Derivatives such as purines and pyrimidines bases in an amount from 3 to 25 g/kg of dry matter,
PUPY content from 1 to 20 g/kg of dry matter,
ratio PUPY/Nucleic Acid Derivatives higher than 20%,
wherein the malt sprouts fraction extract is such as obtained by a method of preparation comprising the following steps:
a. dilution of isolated malt sprouts fractions enriched in anyone of rootlets, acrospires, hulk, dust and mixtures thereof in aqueous phase to obtain diluted malt sprouts fractions;
b. diffusion of these diluted malt sprouts fractions in aqueous phase followed by a finishing step at pH<6 or >8 and a thermal treatment at 100° C. to obtain two phases, an aqueous one and a solid one;
c. separation of the aqueous phase from the solid phase to recover the aqueous phase;
d. optionally microfiltration of the aqueous phase on plate filters to remove fine insoluble particles with a lower size than 1 μm;
e. optionally, treatment of the aqueous phase obtained in step (c) or (d) in order to obtain the desired % of dry matter by total weight of malt sprouts fraction extract;
f. and optionally a sterilization step,
or,
comprising the following steps:
a. dilution of isolated malt sprouts fractions enriched in anyone of rootlets, acrospires, hulk, dust and mixtures thereof in aqueous phase to obtain diluted malt sprouts fractions;
b. hydrolysis of these diluted malt sprouts fractions in the presence of exogenous enzymes, to obtain two phases, an aqueous one and a solid one;
c. separation of the aqueous phase from the solid phase to recover the aqueous phase;
d. optionally microfiltration of the aqueous phase on plate filters to remove fine insoluble particles with a lower size than 1 μm;
e. optionally, treatment of the aqueous phase obtained in step (c) or (d) in order to obtain the desired % of dry matter by total weight of malt sprouts fraction extract;
f. and optionally a sterilization step,
or,
comprising the following steps:
a. dilution of isolated malt sprouts fractions enriched in anyone of rootlets, acrospires, hulk, dust and mixtures thereof in aqueous phase to obtain diluted malt sprouts fractions;
b. hydrolysis of these diluted malt sprouts fractions in the presence of exogenous enzymes, followed by a finishing step at pH<6 or >8 and thermal treatment at 100° C. to obtain two phases, an aqueous one and a solid one;
c. separation of the aqueous phase from the solid phase to recover the aqueous phase;
d. optionally microfiltration of the aqueous phase on plate filters to remove fine insoluble particles with a lower size than 1 μm;
e. optionally, treatment of the aqueous phase obtained in step (c) or (d) in order to obtain the desired % of dry matter by total weight of malt sprouts fraction extract;
f. and optionally a sterilization step.

38. Composition consisting of malt sprouts fraction extract according to claim 37, wherein the malt sprouts fractions comprise:
dry matter by total weight of the malt sprouts from 92% to 98%,
crude protein content from 22.5 to 37 g/100 g of malt sprout fraction
free Amino Nitrogen content higher than 200 mg/100 g of dry matter.

39. Composition consisting of malt sprouts fraction extract according to claim 37, wherein the malt sprouts fractions comprise:
dry matter by total weight of the malt sprouts from 92% to 98%,
crude protein content from 22.5 to 37 g/100 g of malt sprout fraction
free Amino Nitrogen content higher than 200 mg/100 g of dry matter,
and containing:
total fibers content from 43 to 52 g/100 g of dry matter,
total carbohydrates content from 7 to 12 g/100 g of dry matter,
total sugars content from 4 to 6 g/100 g of dry matter,
fats content below 1 g/100 g,
ash content from 5 to 7 g/100 g.

40. Composition according to claim 30, further comprising at least one ingredient selected from additional ingredients.

41. Composition according to claim 30, further comprising at least one ingredient selected from protein hydrolysates, organic acids, mineral acids, mineral salts, sugars, polyols and mixtures thereof, or protein hydrolysates originating from soy, rapeseed, pea, alfalfa, wheat protein, casein, gelatin, or meat

42. Composition according to claim 30, further comprising at least one ingredient selected from protein hydrolysates, organic acids, mineral acids, mineral salts, sugars, polyols and mixtures thereof, or protein hydrolysates originating from soy, rapeseed, pea, alfalfa, wheat protein, casein, gelatin, or meat, comprising:
total Nitrogen content from 45 to 140 g/kg of dry matter
free Amino Nitrogen content from 10 to 60 g/kg of dry matter, and
nucleic Acid Derivatives such as purines and pyrimidines bases in an amount from 2 to 10 g/kg of dry matter,
PUPY content from 0.5 to 8 g/kg of dry matter,
ratio PUPY/Nucleic Acid Derivatives higher than 20%.

43. Composition according to claim 30, further comprising at least one ingredient selected from protein hydrolysates, organic acids, mineral acids, mineral salts, sugars, polyols and mixtures thereof, or protein hydrolysates originating from soy, rapeseed, pea, alfalfa, wheat protein, casein, gelatin, or meat, comprising:
total Nitrogen content from 45 to 140 g/kg of dry matter,
free Amino Nitrogen content from 10 to 60 g/kg of dry matter, and
nucleic Acid Derivatives such as purines and pyrimidines bases in an amount from 2 to 10 g/kg of dry matter,
PUPY content from 0.5 to 8 g/kg of dry matter,
ratio PUPY/Nucleic Acid Derivatives higher than 20%, said composition being a food ingredient, or a flavor enhancer, used in food for animals or humans, or a culture and/or fermentation medium.

44. Method of preparing a culture and/or performing a fermentation process, comprising a step of adding a composition containing malt sprouts fraction extract, wherein the composition comprises at least 0.5%, and less than 98% of dry matter by total weight of the composition, and comprising:
total Nitrogen content from 40 to 140 g/kg of dry matter,
free Amino Nitrogen content from 6 to 60 g/kg of dry matter, and
nucleic Acid Derivatives such as purines and pyrimidines bases in an amount from 3 to 25 g/kg of dry matter,
PUPY content from 1 to 20 g/kg of dry matter,
Ratio PUPY/Nucleic Acid Derivatives higher than 20%.

45. Method according to claim 44, wherein the fermentation process comprises adding mycetes including actinomycetes, fungi yeasts and/or bacteriae.

46. Method according to claim 44, for the production of metabolites by said fermentation process.

47. Method according to claim 44, wherein the culture comprises microbial cells, plant cells and/or animal cells.

48. Method according to claim 44, wherein the culture comprises a living micro-organism; and comprising at least a step wherein said composition is added to the culture and/or fermentation medium or replaces it as a substitute.
